(19) United States
(12) Patent Application Publication
(10) Pub. No.: US 2016/0010109 A1
(43) Pub. Date: Jan. 14, 2016

(54) ENHANCED ADAPTATION OF CORN

(71) Applicant: PIONEER HI BRED INTERNATIONAL, INC, Johnston, IA (US)

(72) Inventors: Marc C. Albertsen, GRIMES, IA (US); XIAOMU NIU, JOHNSTON, IA (US); DONGSHENG FENG, URBANDALE, IA (US); STEVEN PAUL KING, STRATFORD (CA); DWIGHT TOMES, GRIMES, IA (US)

(21) Appl. No.: 14/771,009

(22) PCT Filed: Mar. 13, 2014
(86) PCT No.: PCT/US14/26279

§ 371 Date: Aug. 27, 2015

Related U.S. Application Data
(60) Provisional application No. 61/781,199, filed on Mar. 14, 2013.

(51) Int. Cl. C12N 15/82; A01H 5/10; G01N 33/00; A01H 1/04

(52) U.S. Cl. CPC C12N 15/8273; A01H 1/04; A01H 5/10; G01N 33/0098

(57) ABSTRACT

Methods and compositions to adapt corn to grow in a geographical location such as northern dry climatic region are disclosed. By modulating phenotypic parameters such as for example flowering time, plant architecture, abiotic stress tolerance in a modular approach, corn is modified to grow in a geographical location that generally does not support growth corn to generate higher yields. Various methods, genes, and compositions are disclosed to improve adaptability and productivity of corn in the desired climatic conditions.

### FIELD

The field of disclosure relates to plant breeding and genetics and, in particular, relates to recombinant DNA constructs useful in plants for enhanced adaptation of corn.

### BACKGROUND

Improving agronomic traits in crop plants is beneficial to farmers. Corn productivity depends on a number of parameters including moisture, temperature, length of the growing season, plant architecture and agronomic practices.

Corn growing conditions vary depending on the soil type, geographical location and other environmental conditions. Generally, optimal average temperatures for corn are around 70° F. and they vary over the corn growing season and during daytime and nighttime. However, overall, corn growth is preferred in warmer climate. Similarly, while corn can survive short exposure to both low and high temperatures e.g., higher than 100° F. or below 32° F., both the high and cold temperatures slow down growth. Extremely low temperatures cause freezing damage and ultimately plant death depending on the duration and the growth stage of the plant. Freezing or frost conditions upon germinating seedlings impact growth. For example, extended low temperatures at seedling stage where the soil temperatures remain below freezing can kill corn. A long exposure of late growth stage corn to temperatures below 30° F. can damage the “growing point”. Low soil temperatures may also result in poor germination and poor standability.

Corn productivity also depends on the length of growing season, which is generally characterized by the Growing Degree Day (GDD) accumulations (commonly referred to as Growing Degree Units (GDUs) or CHU (Crop Heat Units), or heat units (HUs)). The GDD is accumulated from the day after planting until physiological maturity. The GDD calculation for corn is generally well known.

Flowering time determines maturity and that is an important agronomic trait. Genes that control the transition from vegetative to reproductive growth are essential for manipulation of flowering time. Flowering genes will provide opportunities for enhanced crop yield, adaptation of germplasm to different climatic zones and synchronous flowering for hybrid seed production. Developing early-flowering inbred lines will facilitate the movement of elite germplasm across maturity zones.

Natural responses to abiotic stress vary among plant species and among varieties and cultivars within a plant species. Certain species, varieties or cultivars are more tolerant to abiotic stress such as drought than others. Transgenic approaches including overexpression and downregulation are evaluated for engineering drought or cold tolerance in crop plants. Nitrogen utilization efficiency also affects crop yield, especially where the application of nitrogen fertilizer is limited.

### SUMMARY

Methods and compositions to adapt corn to grow in a climatic zone considered not ideal for corn are disclosed herein.

A method of increasing yield by adapting corn plant to grow in a crop-growing environment characterized as northern continental dry climatic region having an average annual CHU of about 1700 to 2000 when measured in ° F. or an average annual GDU of about 1400 to about 1700 when measured in ° F., the method includes expressing one or more recombinant nucleic acids conferring a frost tolerant phenotype when the plant is exposed to −3° C. for about 3 hours; and expressing one or more recombinant nucleic acids that reduce the comparative relative maturity of corn to about 60-70 or wherein a reduction of about 4-10 days in maturity is achieved when compared to a control plant not having the recombinant nucleic acids; and increasing the yield of corn to an average yield of at least about 100 bu/acre.

In an embodiment, the corn plant further includes a recombinant nucleic acid that increases harvest index and optionally reduces the plant stature including plant height. In an embodiment, the corn plant is capable of being planted at a higher population density compared to corn plants not comprising the recombinant nucleic acid. In an embodiment, the corn plant is chilling tolerant after being exposed to temperatures of less than about 15° C. In an embodiment, the corn plant is exposed to frost conditions during a seedling stage. In an embodiment, the corn plant is exposed to frost during grain filling stage. In an embodiment, the corn plant further includes a modified plant architecture or change in harvest index through the modulation of one or more transgenes. In an embodiment, the modified plant architecture includes a modification selected from the group consisting of increased harvest index, shorter stature, reduced leaf angle, and reduced canopy.

In an embodiment, the relative maturity of corn is reduced by modulating a maturity parameter selected from the group consisting of flowering time, grain filling and senescence. In an embodiment, the nucleic acids involved in affecting flowering time include for example, those selected from the group consisting of FTM1, Rap2.7, ZAP1, ZCN8 or a gene involved in floral transition.

In an embodiment, the corn plants described herein are planted at a planting density of about 20,000 plants to about 50,000 plants per acre. For example, planting densities of about 18,000, 22,000, 24,000, 25,000, 28,000, 30,000, 32,000, 34,000, 36,000, 38,000, 40,000 and 42,000 are contemplated.

In an embodiment, the frost tolerance phenotype is conferred by transgenic modulation of one or more nucleic acids that provide chilling or frost tolerance. In an embodiment, the plant architecture is modified by transgenic modulation of one or more nucleic acids selected from the group consisting of maturity reducing genes, dwarfing genes, growth suppressing genes, moderated dwarfing genes and Della proteins or a gene involved in biosynthesis, metabolism of and response to phytohormone Gibberellic acid (GA). In an embodiment, the corn does not exhibit negative agronomic characteristics such as root lodging or stalk lodging due to early maturity.

In an embodiment, the corn plants described herein further include a genetic modification for premature senescence.

A method of increasing yield by adapting corn plant to grow in a crop-growing environment, the method includes expressing one or more recombinant nucleic acids conferring a frost tolerant phenotype when the corn plant is exposed to about −3° C. for about 3 hours; selecting a genetic modification that reduces the comparative relative maturity of the corn plant to about 60-70 days or wherein a reduction of about 7-10 days is achieved in the corn plant when compared to a control corn plant not having the genetic modifications and increasing the yield of corn to at least about 100 bu/acre.

In an embodiment, the genetic modifications described herein include marker-assisted breeding. In an embodiment, the genetic modification includes a single nucleotide polymorphism (SNP) marker. In an embodiment, the genetic modification includes a quantitative trait locus.

A method of crop rotation in a crop growing field for barley, wheat, corn, and brassica in a crop-growing environment characterized as northern continental dry climatic region having an average annual CHU of about 1700 to 2000 when measured in ° F. or an average annual GDU of about 1400 to about 1700 when measured in ° F., the method includes growing brassica or barley or wheat in a first crop growing season in a field within the northern continental dry climatic region; growing corn in the field in a second crop growing season, wherein the corn is transgenically modified to tolerate frost when exposed to −3° C. for about 3 hours and the corn further includes one or more genetic modifications that reduce the comparative relative maturity of corn to about 60-70 days; and rotating the brassica or barley or wheat crop with the corn in the field. In an embodiment, the crop rotation follows a pattern of barley-corn-barley or corn-brassica-corn. In an embodiment, the corn crop in the field is followed by a spring canola crop in the field.

A method of screening for corn plants that are tolerant to freezing, the method includes acclimatizing corn seedlings at about V2-V4 stage at about 8-12° C. for about 4-6 hours followed by a cold treatment at about 3-5° C. for about 14-18 hours under no light; treating the acclimatized seedlings to a freezing condition of about −2° C. to −3° C. for about 3-6 hours depending on the genotype of the seedlings; transferring the seedlings to room temperature; and screening the seedlings for survival after 3-5 days. In an embodiment, the seedling is a transgenic seedling that includes a recombinant nucleic acid. In an embodiment, wherein the seedling includes a marker associated with freezing tolerance. In an embodiment, the screening method includes assigning a binary value for survival or death of the seedlings. In an embodiment, the cold acclimatization of the seedlings is performed in a growth chamber.

A method of screening for corn plants that are tolerance to freezing during a reproductive growth stage, the method includes acclimatizing one or more corn plants at about R3-R4 stage at about 8-12° C. for about 4-6 hours; treating the acclimatized corn plants to a freezing condition of about −2° C. to −3° C. for about 1 hour depending on the genotype of the seedlings; transferring the corn plants to room temperature; and measuring a photosynthetic parameter at one of 1, 5, and 24 hours after the freezing treatment. In an embodiment, the corn plant is a transgenic seedling comprising a recombinant nucleic acid. In an embodiment, the corn plant contains a marker associated with freezing tolerance. In an embodiment, the photosynthetic parameter measured is chlorophyll fluorescence. In an embodiment, the corn plant is an inbred. In an embodiment, the corn plant that is screened for freezing or chilling or cold tolerance is a hybrid.

A method of obtaining a corn plant that is adapted to a growing environment characterized as a northern continental dry climatic region having an average annual CHU of about 1700 to 2000 when measured in ° F. or an average annual GDU of about 1400 to about 1700 when measured in ° F., the method includes generating a corn plant having one or more recombinant nucleic acids conferring a frost tolerant phenotype when exposed to −3° C. for about 3 hours; identifying one or more genetic variations or those that are in association with said genetic variations that reduce the comparative relative maturity of corn to about 60-70 days; and obtaining the corn plant having the one or more recombinant nucleic acids and the genetic variations. In an embodiment, the corn plant has a yield of at least about 100 bu/acre.

A method of reducing the flowering time in a field population of corn plants, the method includes growing a population of corn plants in a geographical region, wherein the relative maturity of the corn plants is higher compared to the corn plants normally grown in the geographical region; and modifying the relative maturity of one of the corn plants by an exogenous application of a nucleic acid material such that the relative maturity of the corn plants is substantially reduced to the maturity level desired for the geographical region. In an embodiment, the nucleic acid material is a single stranded DNA, single stranded RNA, dsRNA or dsDNA. In an embodiment, the nucleic acid material selectively suppresses one or more nucleic acids involved in flowering time regulation. In an embodiment, the nucleic acid material selectively enhances grain filling or promotes senescence.

A corn plant comprising a frost tolerant phenotype when exposed to −3° C. for about 3 hours and further includes in its genome one or more recombinant nucleic acids, wherein the expression of the nucleic acids reduce the comparative relative maturity of the corn plant to about 60-70 days or wherein a reduction of about 7-10 days is achieved when compared to a control plant not having the recombinant nucleic acids when grown in a region having an average annual CHU of about 1700 to 2000 when measured in ° F. or an average annual GDU of about 1400 to about 1700 when measured in ° F. In an embodiment, the corn plant comprises a modified plant architecture. In an embodiment, the modified plant architecture comprises a modification selected from the group consisting of increased harvest index, shorter stature, reduced leaf angle and reduced canopy. In an embodiment, the relative maturity of corn plant is reduced by modulating a maturity parameter selected from the group consisting of flowering time, grain filling, and senescence.

Seeds or grains are produced from the corn plants described herein. A corn plant having a reduced relative maturity of 60-70 days and further comprising in its genome one or more recombinant nucleic acids, wherein the expression of the nucleic acids provide a frost tolerant phenotype when exposed to −3° C. for about 3 hours and when grown in a region having an average annual CHU of about 1700 to 2000 when measured in ° F. or an average annual GDU of about 1400 to about 1700 when measured in ° F.

In an embodiment, the frost tolerance phenotype is provided by the expression of a transcription factor. In an embodiment, the relative maturity of the corn plant is reduced by modulating a maturity parameter selected from the group consisting of flowering time, grain filling, and senescence. In an embodiment, the relative maturity of corn is reduced by the expression of a nucleic acid to induce RNA interference in the corn plant. In an embodiment, the relative maturity of corn is reduced by the expression of a flowering time regulation gene.

A method of disease or pest management in a crop-growing environment characterized as northern continental dry climatic region having an average annual CHU of about 1700 to 2000 when measured in ° F. or an average annual GDU of about 1400 to about 1700 when measured in ° F., the method includes growing a corn crop in a first crop growing season with a population of corn plants that exhibit a frost tolerant phenotype when exposed to −3° C. for about 3 hours and comprising in its genome one or more recombinant nucleic acids, wherein the expression of the nucleic acids reduce the comparative relative maturity of the corn plant to about 60-70 days or wherein a reduction of about 7-10 days is achieved when compared to a control plant not having the recombinant nucleic acids; and rotating the corn crop with a barley crop or wheat or a brassica crop in a second growing season and thereby controlling the disease or pest infestation in the crop-growing environment. In an embodiment, the pests are insect pests. In an embodiment, the corn crop is rotated with a barley or wheat or brassica crop after two consecutive corn crops to reduce pest resistance incidence.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows a schematic of various stresses and growth stage during the development of corn in a northern dry climatic region of interest and how they affect maturity.

### SUMMARY OF SEQ ID NOS

| Description and Abbreviation | SEQ ID NO: |
|---|---|
| Maize FTM1 amino acid sequence (flowering time | 1 |
| regulation) | |
| Maize FTM1 coding DNA sequence (flowering time | 2 |
| regulation) | |
| Maize UBI promoter | 3 |
| Rice ACTIN promoter with 5′-UTR and Intron 1 | 4 |
| ZM-RAP2.7 peptide (flowering time regulation) | 5 |
| ZM-RAP2.7 coding DNA sequence (flowering time | 6 |
| regulation) | |
| ZM-ZAP1 peptide (flowering time regulation) | 7 |
| ZM-ZAP1 coding DNA sequence (flowering time | 8 |
| regulation) | |
| ZM-SEE1 PRO with ADH1 Intron1 | 9 |
| ZM-SGR1 peptide (flowering time regulation) | 10 |
| ZM-SGR1 coding DNA sequence (flowering time | 11 |
| regulation) | |
| ZM-PRE-ES peptide (early senescence) | 12 |
| ZM-PRE-ES coding DNA sequence (early senescence) | 13 |
| RAB17 promoter sequence | 14 |
| ZM-NPK1B peptide (frost tolerance - signal | 15 |
| transduction) | |
| ZM-NPK1B coding DNA sequence (frost tolerance- | 16 |
| signal transduction) | |
| ZM-LIP15 promoter sequence | 17 |
| TA-DREB3 peptide (frost tolerance- signal | 18 |
| transduction) | |
| TA-DREB3 DNA coding sequence (frost tolerance- | 19 |
| signal transduction) | |
| AT-CBF2 peptide (frost tolerance- signal transduction) | 20 |
| ZM-SPX1 peptide (frost tolerance- signal transduction) | 21 |
| ZM-SPX1 DNA coding sequence (frost tolerance- | 22 |
| signal transduction) | |
| ZM-DGAT1-2 (ASK) peptide (frost tolerance- | 23 |
| membrane integrity) | |
| ZM-DGAT1-2 (ASK) coding DNA sequence (frost | 24 |
| tolerance-membrane integrity) | |
| MS-S2A promoter sequence | 25 |
| ZM-D8MPL peptide (Architecture modification- stature | 27 |
| reduction) | |
| ZM-D8MPL coding DNA sequence (Architecture | 28 |
| modification- stature reduction) | |
| SB-EUI1 peptide (Architecture modification- stature | 29 |
| reduction) | |
| SB-EUI1 DNA coding sequence (Architecture | 30 |
| modification- stature reduction) | |
| ZM-LG1 peptide (Architecture modification - Leaf | 31 |
| angle) | |
| ZM-LG1 DNA coding sequence (Architecture | 32 |
| modification - Leaf angle) | |
| ZM-ADF4 PRO with 5′-UTR and Intron 1 | 33 |
| ZM-DWF4 peptide (Architecture modification - Leaf | 34 |
| angle) | |
| ZM-DWF4 DNA coding sequence (Architecture | 35 |
| modification - Leaf angle) | |
| ZM-FTM1 PRO | 36 |
| ZM-MIR156B (non-coding RNA; Architecture | 37 |
| modification-canopy alteration) | |

The sequence descriptions and Sequence Listing attached hereto comply with the rules governing nucleotide and/or amino acid sequence disclosures in patent applications as set forth in 37 C.F.R. §1.821-1.825. The sequence listing is hereby incorporated by reference.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219(2):345-373 (1984) which are herein incorporated by reference. The symbols and format used for nucleotide and amino acid sequence data comply with the rules set forth in 37 C.F.R. §1.822.

### DETAILED DESCRIPTION

Early flowering-increase the length of time available for grain fill/maturation by inducing hybrids to flower earlier in development and/or shorten the time required for grain fill duration/maturation. Suitable target reduction in the days for maturity described herein includes about 5-15 CRM or 5-7 CRM, 7-10 CRM or 10-15 CRM.

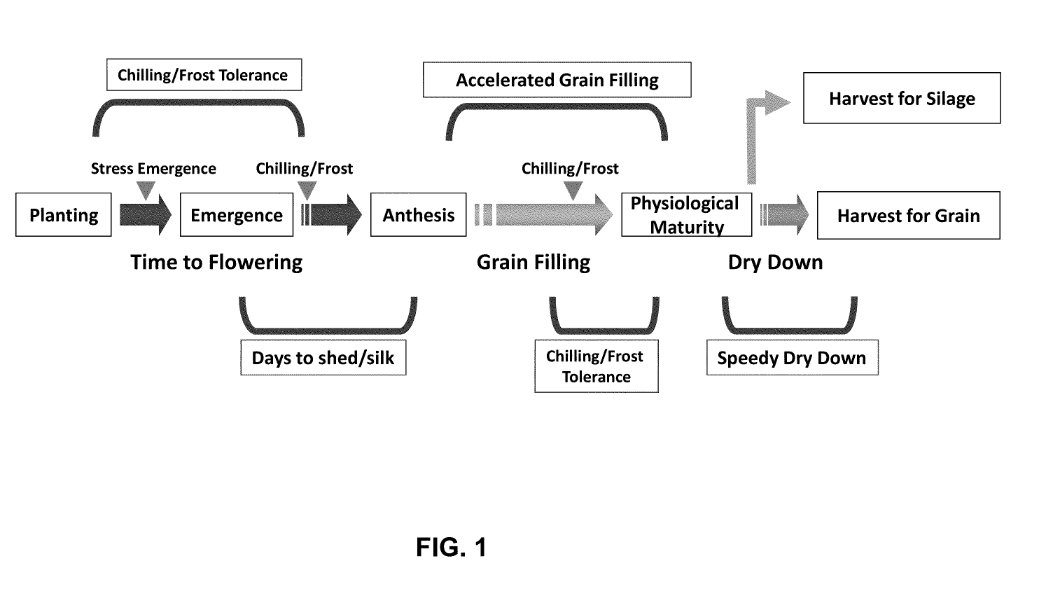

FIG. 1 illustrates several various components related to the modulation of overall corn maturity. Maturity generally refers to the duration between the planting of seeds to harvesting grains. During this process, plants go through three major stages—time to flowering, grain filling and dry down. Time to flowering includes seed planting, emergence through anthesis—all of which are vegetative growth. During this stage, plants accumulate biomass and establish canopy growth. Grain filling is the second main stage, when plants are actively depositing photosynthates into growing grains from post-anthesis to physiological maturity. The transfer of sugars between sources (photosynthetic leaves) and sink (ears) is fundamental for grain yield. The last stage of dry down is specific for grain corn. Unlike silage corn, which can be harvested at physiological maturity without drying, grain corn can be mechanically harvested with grain moisture content below around 20%.

Since maturity includes all 3 stages, shortening any one or more stages would result in an overall reduction in maturity. One or more of the following technical approaches achieve shortened maturity: reducing days to shed and silk (flowering), accelerating grain filling or decreasing duration for dry down. In addition, grain yield risks due to chilling and frost damage are shown in FIG. 1. Stages when corn plants are most prone to low temperature stress are at emergence, often referred to as stress emergence; early seedling growth, and mid- to late-season during grain filling. Tolerance at these stages helps safeguard a healthy plant canopy, and help achieve a fully realized grain yield.

Frost tolerance confers the ability of the maize plant to resist damage from mild frost occurrences. Suitable target includes about 3 hours at −3° C. or −2° C. for 4 hours or a range of 0° C. to about −5° C. for about 2-5 hours. Cold/chilling tolerance provides the ability of the maize plants to more rapidly recover and/or resist tissue damage from a high light chilling event, for example on cold bright mornings. Suitable target is recovery of photosynthetic capacity within 24 hrs following exposure <15° C. or longer periods e.g., 36, 48 hours when exposed to colder temperatures of less than 10° C. or 5° C.

Increased yield and reduction of above ground biomass (e.g., dwarfing) allows for increasing harvest index of about 20% and is targeted to increase grain yield per acre through enabling increased planting densities. Shorter stature may also reduce residue in colder northern environments that is prone to slower breakdown of the residue.

Germination tolerance is a valuable trait in conditions where stress during seedling emergence can be detrimental to crop yield due to lower soil temperatures. Stress emergence score of 4-5 is adequate for the northern continental dry climatic regions described herein. In addition to germination tolerance, lower evaporative/lower evapotranspiration, shorter season environment may help achieve 100 bu/acre.

An early frost during the grain-filling period can cause losses in corn yield and quality depending on the temperature, duration, and corn growth stage at the time of the frost. For example, a severely damaging frost may occur at 32° F. for 4 to 5 hours or 28° F. for only 5 to 10 minutes that can kill the entire corn plant or severely damage the leaves, stalk, ear shank and husks. A light frost of 30 to 32° F. for 1 or 2 hour can kill corn leaves, but not the corn stalk. Damaging frost can occur at slightly above 32° F. and the ideal conditions for rapid heat loss from the corn leaves. Leaf temperature can drop below actual air temperature which generally only results in damage to the uppermost leaves of the corn plant.

Heat units (HU) are used to explain temperature impact on rate of corn development, and these HUs provide growers an indexing system for selection of corn hybrids in a given location. Several formulas exist for the calculation of heat units. Among them, GDD or GDU (Growing Degree Day or Growing Degree Unit) and CHU (Crop Heat Units) are most commonly used. GTI (General Thermal Index) has recently been developed that attempts to improve accuracy in predicting developmental stages.

GDDs, also known as GDUs, are often referred to simply as HUs in the US. The method to calculate GDD is to average daily temperature (degrees F.) then minus 50, proposed by the National Oceanic and Atmospheric Administration and labeled as the “Modified Growing Degree Day”.

GDU=(Tₘₐₓ+Tₘᵢₙ)2−T_{base}

Where Tₘₐₓ is maximum daily temperature, Tₘᵢₙ is minimum daily temperature, and Tbase is a base temperature (mostly set at 50 F).

CHUs are first developed and used in Ontario, Canada in the 1960's. The method to calculate CHU is somewhat more complex, allocating different responses of development to temperature (degrees C.) between the day and the night.

CHU_{day}=3.33*(Tₘₐₓ−10)−0.084*(Tₘₐₓ−10)2

CHU_{night}=1.8*(Tₘᵢₙ−4.4)

CHU=[CHU_{day}+CHU_{night}]/2

GTIs are calculated based on different responses of corn from planting to silking and from silking to maturity. The period between planting and silking is defined as vegetative growth, whereas time from silking to maturity is the grain filling stage.

F_{T(veg)}=0.0432T²−0.000894T³

F_{T(fill)}=5.358+0.011178T²

GTI=F_{T(veg)}+F_{T(fill)}

Where T is mean daily temperature (degrees C.), F_{T(veg)} is for the period from planting to silking, F_{T(fill)} is for the period from silking to maturity.

Relative Maturity Conversion Guidelines

Guidelines for converting various relative-maturity rating systems have been reported by Dwyer, et al., (Agron. J. 91:946-949). Conversions for CHU, GDD and the Corn Relative Maturity rating system (CRM), also referred to as the Minnesota Relative Maturity Rating, are generally available. The CRM rating system is widely used in the US to characterize hybrid relative maturity. The CRM rating is not based on temperature, but on the duration in days from planting to maturity (in an average year) relative to a set of standard hybrids. The approximate conversion from one rating system to another can be estimated from a linear regression equation. Some data sets calculate GDDs from degree Fahrenheit, resulting in a number that is 1.8× larger than that when using degree Celcius in the estimation of CHU or CRM from GDD (or 1.8× smaller when estimating GGD from CHU or CRM). (University of Guelph Publication; Corn Maturity and Heat Units, can be accessed via plant.uoguelph.ca/research/homepages/ttollena/research/cropheatunits.html, using the prefix www).

Maturity may also generally refer to a physiological state, where maximum weight per kernel has been achieved for the planted corn. This is often referred to as physiological maturity and is generally associated with the formation of an abscission layer or “black layer” at the base of the kernel. One of the most commonly used methods for designating hybrid maturity ratings (days to maturity) is based on comparisons among hybrids close to the time of harvest.

Kernel dry weight does not generally increase beyond physiological maturity. Kernel drying that occurs following black layer is mostly due to evaporative moisture loss. Drydown rates are generally the greatest during the earlier, warmer part of the harvest season and decline as the weather gets colder.

Corn as disclosed herein matures earlier and will dry down faster due to more favorable drying conditions early in the harvest season than in the later part of the season where it gets colder. Dry down during colder temperatures is slower. Corn drydown rate is generally linked to daily growing degree unit (GDU) accumulation and because GDU accumulation can vary widely during the harvest season, early maturity corn as disclosed herein enable planting early during the season and harvesting early during the growth season that is generally short in the northern dry continental climatic regions.

Some of the characteristics that affect dry down of the corn plants disclosed herein include husk leaf coverage, leaf number, husk leaf senescence, ear angle and kernel pericarp characteristics.

Harvest index, the ratio of the grain to total aboveground biomass, is an indicator of dry matter partitioning efficiency. It has remained generally around 50% in conventional maize. In comparison to maize, harvest index acquired a different role in increasing plant standability in small grain cereals where it was significantly increased with the introduction of dwarfing genes. Reduced stature made these cereals less likely to lodge by reducing torque on the top-heavy straw, which allowed for higher inputs such as fertilizers and irrigation, resulting in increased biomass production per unit land area. Whereas yield increases in small grain cereals have resulted from an increase in both harvest index and total biomass production per unit land area, those in maize have been the consequence of mainly an increase in total biomass. Increased planting density as a means of increasing grain yield in maize has affected changes in leaf angle and shape as adaptations to this environment and has in general resulted in increased plant and ear heights. The stalk becomes mechanically weaker with increasing planting density because of reduction in individual plant vigor that results from a nonlinear relationship between planting density and biomass increase. The methods and compositions disclosed herein provide improved plant architecture and reduced root/stalk lodging as compared to control plants not having the transgene or not having the genetic modifications. Cellulose synthases to improve stalk strength including mid-season snap or late-season lodging are disclosed, for example, in U.S. Pat. No. 8,207,302, incorporated by reference with respect to the cellulose synthase (Ces) sequences disclosed therein.

A method of increasing yield by adapting corn plant to grow in a crop-growing environment characterized as northern continental dry climatic region having an average annual CHU of about 1700 to 2000 when measured in ° F. or an average annual GDU of about 1400 to about 1700 when measured in ° F., the method includes expressing one or more recombinant nucleic acids conferring a frost tolerant phenotype when the plant is exposed to −3° C. for about 3 hours; and expressing one or more recombinant nucleic acids that reduce the comparative relative maturity of corn to about 60-70 days or wherein a reduction of about 4-10 days is achieved when compared to a control plant not having the recombinant nucleic acids; and increasing the yield of corn to an average yield of at least about 100 bu/acre.

The term northern continental dry climatic region generally refers to a geographical region that is characterized by colder than normal temperatures in the summer compared to normal corn growing areas and shorter growing seasons with lower than normal precipitation, compared to for example, the corn belt of the mid-west United States, such as for example, the state of Iowa. In an embodiment, such regions are characterized those having an average annual CHU of about 1700 to 2000 when measured in ° F. or an average annual GDU of about 1400 to about 1700 when measured in ° F. In an embodiment, an average annual CHU of about 1650 to 2200 when measured in ° F. or an average annual GDU of about 1350 to about 1850 when measured in ° F. In an embodiment, an average annual CHU of about 1650 to 2200 when measured in ° F. or an average annual GDU of about 1350 to about 1850 when measured in ° F. In an embodiment, an average annual CHU of about 1750 to 1900 when measured in ° F. or an average annual GDU of about 1500 to about 1600 when measured in ° F. Any variation in the calculation of GDUs or CHUs or GDDs depending on parameters used, e.g. ° F. or ° C., is within the scope of this disclosure. GDU, CHU, GDD calculations can be made using tools available to one ordinary skill in the art.

Corn plants or hybrids disclosed herein further include improved standability where significant field drying is expected. Traits generally associated with improved hybrid standability such as for example, resistance to stalk rot and leaf blights, genetic stalk strength (a thick stalk rind), short plant height, lower ear placement and high late-season plant health are within the scope of the methods and compositions disclosed herein.

In an embodiment, the corn plant further includes a recombinant nucleic acid that increases harvest index and optionally reduces the plant stature including plant height. In an embodiment, the corn plant is capable of being planted at a higher population density compared to corn plants not comprising the recombinant nucleic acid. In an embodiment, the corn plant is chilling tolerant after being exposed to temperatures of less than about 15° C. Chilling tolerance at either lower or higher temperatures are also contemplated, for example at 4, 6, 8, 10, 12, 18° C.

In an embodiment, the corn plant is exposed to frost conditions during a seedling stage. The seedling stage stress could be at emergence, due to early planting under seasonably cooler conditions. With below average temperatures in the growing season, corn seeds may be in the ground for three weeks or more before seedlings emerge. The growth stage designated as VE generally refers to emergence and the vegetative stages are generally referred to as V1, V2, V3, V4 and other V stages until tassel emergence (VT).

In an embodiment, the corn plant is exposed to frost during grain filling stage. The reproductive stages are often referred to as R1, R2, R3 and other R stages. R1 is the first reproductive stage and will generally occur about two to three days after VT. R1 occurs when silks have emerged from the tip of the ear shoot on at least 50% of the plants. R2 or the blister stage generally occurs about 10-14 days after silking and the kernel filling occurs. Stress during reproductive stage such as R2 or R3 may result in kernel abortion.

In an embodiment, the corn plant further includes a modified plant architecture or change in harvest index through the modulation of one or more transgenes. In an embodiment, the modified plant architecture includes a modification selected from the group consisting of increased harvest index, shorter stature, reduced leaf angle, and reduced canopy.

In an embodiment, the relative maturity of corn is reduced by modulating a maturity parameter selected from the group consisting of flowering time, grain filling and senescence. In an embodiment, the nucleic acids involved in affecting flowering time include for example, those selected from the group consisting of FTM1, Rap2.7, ZAP1, ZCN8 or a gene involved in floral transition.

In an embodiment, the corn plants described herein are planted at a planting density of about 20,000 plants to about 50,000 plants per acre. For example, planting densities of about 15,000, 18,000, 22,000, 24,000, 25,000, 28,000, 30,000, 32,000, 34,000, 36,000, 38,000, 40,000 and 42,000 are also contemplated. The row width range can include 30-inch rows, 24-inch rows, 20-inch rows, 18-inch rows or narrower. The reduced stature of the corn plants disclosed herein is advantageous for narrower row spacing, thereby increasing the planting density.

In an embodiment, the frost tolerance phenotype is conferred by transgenic modulation of one or more nucleic acids that provide chilling or frost tolerance. In an embodiment, the plant architecture is modified by transgenic modulation of one or more nucleic acids selected from the group consisting of maturity reducing genes, dwarfing genes, growth suppressing genes, moderated dwarfing genes and Della proteins or a gene involved in biosynthesis, metabolism of and response to phytohormone Gibberellic acid (GA). In an embodiment, the corn does not exhibit negative agronomic characteristics such as root lodging or stalk lodging due to early maturity.

In an embodiment, the corn plants described herein further include a genetic modification for premature senescence.

A method of increasing yield by adapting corn plant to grow in a crop-growing environment, the method includes expressing one or more recombinant nucleic acids conferring a frost tolerant phenotype when the corn plant is exposed to about −3° C. for about 3 hours; selecting a genetic modification that reduces the comparative relative maturity of the corn plant to about 60-70 or wherein a reduction of at least about 7-10 days is achieved in the corn plant when compared to a control corn plant not having the genetic modifications; and increasing the yield of corn to at least about 100 bu/acre.

In an embodiment, the genetic modifications described herein include marker-assisted breeding. In an embodiment, the genetic modification includes a single nucleotide polymorphism (SNP) marker. In an embodiment, the genetic modification includes a quantitative trait locus.

A method of crop rotation in a crop growing field for barley, corn, and brassica in a crop-growing environment characterized as northern continental dry climatic region having an average annual CHU of about 1700 to 2000 when measured in ° F. or an average annual GDU of about 1400 to about 1700 when measured in ° F., the method includes growing brassica or barley in a first crop growing season in a field within the northern continental dry climatic region; growing corn in the field in a second crop growing season, wherein the corn is transgenically modified to tolerate frost when exposed to −3° C. for about 3 hours and the corn further includes one or more genetic modifications that reduce the comparative relative maturity of corn to about 60-70 days; and rotating the brassica or barley crop with the corn in the field. In an embodiment, the crop rotation follows a pattern of barley-corn-barley or corn-brassica-corn. In an embodiment, the corn crop in the field is followed by a spring canola crop in the field.

A method of screening for corn plants that are tolerant to freezing, the method includes acclimatizing corn seedlings at about V2-V4 stage at about 8-12° C. for about 4-6 hours followed by a cold treatment at about 3-5° C. for about 14-18 hours under no light; treating the acclimatized seedlings to a freezing condition of about −2° C. to −3° C. for about 3-6 hours depending on the genotype of the seedlings; transferring the seedlings to room temperature; and screening the seedlings for survival after 3-5 days. In an embodiment, the seedling is a transgenic seedling that includes a recombinant nucleic acid. In an embodiment, wherein the seedling includes a marker associated with freezing tolerance. In an embodiment, the screening method includes assigning a binary value for survival or death of the seedlings. In an embodiment, the cold acclimatization of the seedlings is performed in a growth chamber.

A method of screening for corn plants that are tolerance to freezing during a reproductive growth stage, the method includes acclimatizing one or more corn plants at about R3-R4 stage at about 8-12° C. for about 4-6 hours; treating the acclimatized corn plants to a freezing condition of about −2° C. to −3° C. for about 1 hour depending on the genotype of the seedlings; transferring the corn plants to room temperature; and measuring a photosynthetic parameter at one of 1, 5 and 24 hours after the freezing treatment. In an embodiment, the corn plant is a transgenic seedling comprising a recombinant nucleic acid. In an embodiment, the corn plant contains a marker associated with freezing tolerance. In an embodiment, the photosynthetic parameter measured is chlorophyll fluorescence. In an embodiment, the corn plant is an inbred. In an embodiment, the corn plant that is screened for freezing or chilling or cold tolerance is a hybrid.

A method of obtaining a corn plant that is adapted to a growing environment characterized as a northern continental dry climatic region having an average annual CHU of about 1700 to 2000 when measured in ° F. or an average annual GDU of about 1400 to about 1700 when measured in ° F., the method includes generating a corn plant having one or more recombinant nucleic acids conferring a frost tolerant phenotype when exposed to −3° C. for about 3 hours; identifying one or more genetic variations or those that are in association with said genetic variations that reduce the comparative relative maturity of corn to about 60-70 days; and obtaining the corn plant having the one or more recombinant nucleic acids and the genetic variations. In an embodiment, the corn plant has a yield of at least about 100 bu/acre.

A method of reducing the flowering time in a field population of corn plants, the method includes growing a population of corn plants in a geographical region, wherein the relative maturity of the corn plants is higher compared to the corn plants normally grown in the geographical region; and modifying the relative maturity of one of the corn plants by an exogenous application of a nucleic acid material such that the relative maturity of the corn plants is substantially reduced to the maturity level desired for the geographical region. In an embodiment, the nucleic acid material is a single stranded DNA, single stranded RNA, dsRNA or dsDNA. In an embodiment, the nucleic acid material selectively suppresses one or more nucleic acids involved in flowering time regulation. In an embodiment, the nucleic acid material selectively enhances grain filling or promotes senescence.

A corn plant comprising a frost tolerant phenotype when exposed to −3° C. for about 3 hours and further includes in its genome one or more recombinant nucleic acids, wherein the expression of the nucleic acids reduce the comparative relative maturity of the corn plant to about 60-70 days or wherein a reduction of about 7-10 days is achieved when compared to a control plant not having the recombinant nucleic acids when grown in a region having an average annual CHU of about 1700 to 2000 when measured in ° F. or an average annual GDU of about 1400 to about 1700 when measured in ° F. In an embodiment, the corn plant comprises a modified plant architecture. In an embodiment, the modified plant architecture comprises a modification selected from the group consisting of increased harvest index, shorter stature, reduced leaf angle and reduced canopy. In an embodiment, the relative maturity of corn plant is reduced by modulating a maturity parameter selected from the group consisting of flowering time, grain filling, and senescence.

Seeds or grains are produced from the corn plants described herein. A corn plant having a reduced relative maturity of 60-70 days and further comprising in its genome one or more recombinant nucleic acids, wherein the expression of the nucleic acids provide a frost tolerant phenotype when exposed to −3° C. for about 3 hours and when grown in a region having an average annual CHU of about 1700 to 2000 when measured in ° F. or an average annual GDU of about 1400 to about 1700 when measured in ° F.

In an embodiment, the frost tolerance phenotype is provided by the expression of a transcription factor. In an embodiment, the relative maturity of the corn plant is reduced by modulating a maturity parameter selected from the group consisting of flowering time, grain filling, and senescence. In an embodiment, the relative maturity of corn is reduced by the expression of a nucleic acid to induce RNA interference in the corn plant. In an embodiment, the relative maturity of corn is reduced by the expression of a flowering time regulation gene.

A method of disease or pest management in in a crop-growing environment characterized as northern continental dry climatic region having an average annual CHU of about 1700 to 2000 when measured in ° F. or an average annual GDU of about 1400 to about 1700 when measured in ° F., the method includes growing a corn crop in a first crop growing season with a population of corn plants that exhibit a frost tolerant phenotype when exposed to −3° C. for about 3 hours and comprising in its genome one or more recombinant nucleic acids, wherein the expression of the nucleic acids reduce the comparative relative maturity of the corn plant to about 60-70 days or wherein a reduction of about 7-10 days is achieved when compared to a control plant not having the recombinant nucleic acids; and rotating the corn crop with a barley crop or a brassica crop in a second growing season and thereby controlling the disease or pest infestation in the crop-growing environment. In an embodiment, the pests are insect pests. In an embodiment, the corn crop is rotated with a barley or brassica crop after two consecutive corn crops to reduce pest resistance incidence.

The disclosure of each reference set forth herein is hereby incorporated by reference in its entirety. Some of the agronomic parameters that correlate with nitrogen use efficiency analysis and/or include for e.g., root dwt (g), root: shoot dwt ratio, shoot dwt (g), shoot nitrogen (mg/g dwt), shoot total nitrogen (mg) and total plant dwt (g). Some of the variables that for nitrogen use efficiency reproductive assay include e.g., anthesis to silking interval (days), days to shed, days to silk, ear area 8 days after silk (sq cm), ear length 8 days after silk (cm), ear width 8 days after silk (cm), max total area, specific growth rate, and silk count.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

Thus, the methods of the invention find use in producing dwarf varieties of crop plants. Dwarf crop plants having improved agronomic characteristics, such as, for example, reduced potential for lodging, increased water-use efficiency, reduced life cycle, increased harvest efficiency and increased yield per unit area are obtained by these methods.

By “dwarf” is intended to mean atypically small. By “dwarf plant” is intended to mean an atypically small plant. Generally, such a “dwarf plant” has a stature or height that is reduced from that of a typical plant by about 5%, 10%, 15%, 20%, 25%, 30%, 35%, 40%, 45%, 50%, 55%, 60% or greater. Generally, but not exclusively, such a dwarf plant is characterized by a reduced stem, stalk or trunk length when compared to the typical plant.

Insect resistance traits such as those commercially available presently or later can be stacked with the corn plants described herein. These include for example, lepidopteran resistant corn, rootworm resistant corn, European corn borer resistant corn, BT11, MIR162, MIR604, DAS-06275-8, DAS-59122-7, TC1507, MON810, MON863, MON88017, MON89034. Herbicide tolerance traits include for example NK603, GA21, DAS-40278-9, T25, dicamba tolerant corn, auxin herbicide tolerant corn, glyphosate tolerant corn and any other mode of action tolerant corn.

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

“Agronomic characteristic” or “agronomic parameter” is a measurable parameter including but not limited to, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence” and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from a mRNA template using the enzyme reverse transcriptase. The cDNA can be single-stranded or converted into the double-stranded form using the Klenow fragment of DNA polymerase I.

“Mature” protein refers to a post-translationally processed polypeptide; i.e., one from which any pre- or pro-peptides present in the primary translation product have been removed.

Nitrogen utilization efficiency (NUE) genes affect yield and have utility for improving the use of nitrogen in crop plants, especially maize. Increased nitrogen use efficiency can result from enhanced uptake and assimilation of nitrogen fertilizer and/or the subsequent remobilization and reutilization of accumulated nitrogen reserves, as well as increased tolerance of plants to stress situations such as low nitrogen environments. The genes can be used to alter the genetic composition of the plants, rendering them more productive with current fertilizer application standards or maintaining their productive rates with significantly reduced fertilizer or reduced nitrogen availability. Improving NUE in corn would increase corn harvestable yield per unit of input nitrogen fertilizer, both in developing nations where access to nitrogen fertilizer is limited and in developed nations where the level of nitrogen use remains high. Nitrogen utilization improvement also allows decreases in on-farm input costs, decreased use and dependence on the non-renewable energy sources required for nitrogen fertilizer production and reduces the environmental impact of nitrogen fertilizer manufacturing and agricultural use. Applied nitrogen levels vary depending on the location, cost, desired yield and other factors. For example, one pound of nitrogen per bushel of expected yield is a general framework for selecting nitrogen application rates for corn. For example, a suitable range would include at least about 50, 60, 70, 80, 90, 100, 110, 120, 130, 140, 150, 160, 170, 180, 190, 200 pounds of nitrogen per acre.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

The percent identity between two amino acid or nucleic acid sequences may be determined by visual inspection and mathematical calculation.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal W method of alignment (Thompson, et al., (1994). Nucleic Acids Research 22:4673-80) with the default parameters (GAP PENALTY=10, GAP LENGTH PENALTY=0.2, DELAY DEVERGENT SEQS(%)=30%, DNA TRANSITION WEIGHT=0.5, PROTEIN WEIGHT MATRIX “Gonnet Series”).

Default parameters for pairwise alignments using the Clustal W method were SLOW-ACCURATE, GAP PENALTY=10, GAP LENGTH=0.10, PROTEIN WEIGHT MATRIX “Gonnet 250”. After alignment of the sequences, using the Clustal W program, it is possible to obtain “percent identity” and “divergence” values by viewing the “sequence distances” table on the same program; unless stated otherwise, percent identities and divergences provided and claimed herein were calculated in this manner.

Alternatively, sequence alignments and percent identity calculations may be determined using a variety of comparison methods designed to detect homologous sequences including, but not limited to, the Clustal V method of alignment (Higgins and Sharp, (1989) CABIOS 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

Alternatively, the percent identity of two protein sequences may be determined by comparing sequence information based on the algorithm of Needleman and Wunsch, (J. Mol. Biol. 48:443-453, 1970) and using the GAP computer program available from the University of Wisconsin Genetics Computer Group (UWGCG). The preferred default parameters for the GAP program include: (1) a scoring matrix, blosum62, as described by Henikoff and Henikoff, (Proc. Natl. Acad. Sci. USA 89:10915-10919 1992); (2) a gap weight of 12; (3) a gap length weight of 4; and (4) no penalty for end gaps.

Other programs used by those skilled in the art of sequence comparison may also be used. The percent identity can be determined by comparing sequence information using, e.g., the BLAST program described by Altschul, et al., (Nucl. Acids. Res. 25:3389-3402 1997). This program is available on the Internet at the web site of the National Center for Biotechnology Information (NCBI) or the DNA Data Bank of Japan (DDBJ). The details of various conditions (parameters) for identity search using the BLAST program are shown on these web sites, and default values are commonly used for search although part of the settings may be changed as appropriate. Alternatively, the percent identity of two amino acid sequences may be determined by using a program such as genetic information processing software GENETYX Ver.7 (Genetyx Corporation, Japan) or using an algorithm such as FASTA. In this case, default values may be used for search.

The percent identity between two nucleic acid sequences can be determined by visual inspection and mathematical calculation, or more preferably, the comparison is done by comparing sequence information using a computer program. An exemplary, preferred computer program is the Genetic Computer Group (GCG®; Madison, Wis.) WISCONSIN PACKAGE® version 10.0 program, “GAP” (Devereux, et al., (1984) Nucl. Acids Res. 12:387). In addition to making a comparison between two nucleic acid sequences, this “GAP” program can be used for comparison between two amino acid sequences and between a nucleic acid sequence and an amino acid sequence. The preferred default parameters for the “GAP” program include: (1) the GCG® implementation of a unary comparison matrix (containing a value of 1 for identities and 0 for non-identities) for nucleotides, and the weighted amino acid comparison matrix of Gribskov and Burgess, (1986) Nucl. Acids Res. 14:6745, as described by Schwartz and Dayhoff, eds., “Atlas of Polypeptide Sequence and Structure,” National Biomedical Research Foundation, pp. 353-358, (1979), or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.2.7, available for use via the National Library of Medicine website, or the WU-BLAST 2.0 algorithm (Advanced Biocomputing, LLC). In addition, the BLAST algorithm uses the BLOSUM62 amino acid scoring matrix, and optional parameters that can be used are as follows: (A) inclusion of a filter to mask segments of the query sequence that have low compositional complexity (as determined by the SEG program of Wootton and Federhen (Computers and Chemistry, 1993); also see, Wootton and Federhen, (1996) Methods Enzymol. 266:554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul, 1990; if the statistical significance ascribed to a match is greater than this E-score threshold, the match will not be reported); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75 or 1e-100.

Standard recombinant DNA and molecular cloning techniques used herein are well known in the art and are described more fully in Sambrook, et al., Molecular Cloning: A Laboratory Manual; Cold Spring Harbor Laboratory Press: Cold Spring Harbor, 1989 (hereinafter “Sambrook”).

The term “consisting essentially of” in the context of a polypeptide sequence generally refers to the specified portion of the amino acid sequence and those other sequences that do not materially affect the basic and novel characteristics of the disclosed sequences herein. For example, in the context of an RNAi sequence, the term consisting essentially generally refers to that portion of the target sequence and those other nucleotide sequences that do not materially affect the binding and suppressing properties of the sequence targets disclosed herein.

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

Isolated Polynucleotides and Polypeptides:

The present disclosure includes the following isolated polynucleotides and polypeptides:

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%_{,} 85%, 86%, 87%, 88%, 89%, 90%, 91%, 92%, 93%, 94%, 95%, 96%, 97%, 98%, 99% or 100% sequence identity, based on the Clustal W method of alignment, when compared to a sequence selected from the group consisting of SEQ ID NOS disclosed in Table 1.

An isolated polypeptide wherein the amino acid sequence is a sequence selected from the group consisting of SEQ ID NOS disclosed in Table 1; by alteration of one or more amino acids by at least one method selected from the group consisting of: deletion, substitution, addition and insertion; and (c) a polypeptide wherein the amino acid sequence of the polypeptide comprises a sequence selected from the group consisting of SEQ ID NOS disclosed in Table 1.

An isolated polynucleotide comprising a nucleotide sequence encoding a polypeptide with drought tolerance activity, wherein the nucleotide sequence is hybridizable under stringent conditions with a DNA molecule comprising the full complement of a sequence selected from the group consisting of SEQ ID NOS disclosed in Table 1.

An isolated polynucleotide comprising a nucleotide sequence encoding a polypeptide with drought tolerance activity, wherein the nucleotide sequence is a sequence selected from the group consisting of SEQ ID NOS disclosed in Table 1; by alteration of one or more nucleotides by at least one method selected from the group consisting of: deletion, substitution, addition and insertion.

Recombinant DNA Constructs: In one aspect, the present disclosure includes recombinant DNA constructs.

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, based on the Clustal W method of alignment, when compared to a sequence selected from the group consisting of SEQ ID NOS disclosed in Table 1; or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide encodes a polypeptide.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current disclosure may also be a protein which comprises an amino acid sequence comprising deletion, substitution, insertion and/or addition of one or more amino acids in an amino acid sequence selected from the group consisting of Table 1. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, 10(20):6487-6500, (1982), which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen and those in which a gene is selectively cleaved to remove, substitute, insert or add a selected nucleotide or nucleotides, and then ligated. Alternatively, random mutagenesis approaches may be used to disrupt or “knock-out” the expression of a gene using either chemical or insertional mutagenesis or irradiation. A mutagenesis and mutant identification system known as TILLING (for targeting induced local lesions in genomes) can also be used. In this method, mutations are induced in the seed of a plant of interest, for example, using EMS treatment. The resulting plants are grown and self-fertilized, and the progeny are assessed. For example, the plants may be assed using PCR to identify whether a mutated plant has a mutation, e.g., that reduces expression of a gene. See, e.g., Colbert, et al., (2001) Plant Physiol 126:480-484; McCallum, et al., (2000) Nature Biotechnology 18:455-457.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook, et al., Molecular Cloning: A Laboratory Manual, Third Edition, Chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH₂PO₄, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

The protein of the present disclosure is preferably a protein with drought tolerance activity.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches and small RNA-based approaches.

A suppression DNA construct may comprise a region derived from a target gene of interest e.g., SEQ ID NOS disclosed in Table 1 and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical) to all or part of the sense strand (or antisense strand) of the gene of interest.

For example, an RNAi target sequence includes about 20 to about 1000 contiguous bases of the disclosed SEQ ID NOS disclosed in Table 1 sense or anti-sense strand. In an embodiment, the target sequence includes about 50, 100, 200, 300, 400, 500, 600, 700, 800, 900, 1000, 1100 and 1200 bases of the nucleic acid sequences or amino acids of the protein sequences disclosed herein. Within those contiguous bases, there can be variations and the target RNAi sequences need not be identical and as described above, the similarity level can range from 50% to about 99%.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see, Vaucheret, et al., (1998) Plant J. 16:651-659 and Gura, (2000) Nature 404:804-808).

Another variation describes the use of plant viral sequences to direct the suppression of proximal mRNA encoding sequences (PCT Publication Number WO 1998/36083 published on Aug. 20, 1998).

Promoter inverted repeats are also suitable to suppress the expression of endogenous genes. Such targeted promoter inactivation is possible by identifying the promoter region of endogenous gene and constructing promoter inverted repeat constructs.

Genome editing or genome engineering through site-directed mutagenesis by custom meganucleases with unique DNA-recognition and cleavage properties is possible (e.g., WO 2007/047859 and WO 2009/114321). This technique provides the ability to specifically modify a defined target of interest within a genome. Another site-directed engineering is through the use of zinc finger domain recognition coupled with the restriction properties of restriction enzyme. See, e.g., Urnov, et al., (2010) Nat Rev Genet. 11(9):636-46; Shukla, et al., (2009) Nature 459(7245):437-41. These citations are incorporated herein to the extent they relate to materials and methods to enable genome editing through site-specific modification. Such genome editing techniques are used to engineer site-directed changes including increasing gene expression of an endogenous gene (e.g., placing an enhancer element in control of the transcription), transcriptionally silencing an endogenous gene, creating mutants, variants of the encoded polypeptide, removing one or more genomic regions and other methods to modulate the gene expression and/or its activity.

Knock-out or gene knock-out refers to an inhibition or substantial suppression of endogenous gene expression either by a transgenic or a non-transgenic approach. For example, knock-outs can be achieved by a variety of approaches including transposons, retrotransposons, deletions, substitutions, mutagenesis of the endogenous coding sequence and/or a regulatory sequence such that the expression is substantially suppressed; and any other methodology that suppresses the activity of the target of interest.

Exogenous application of nucleotides including synthetic nucleotide molecules to induce RNAi-mediated silencing of the endogenous gene is possible. See e.g., US 2008/0248576, US 2011/0296556 and WO 2011/112570. Exogenously applied agents are capable of inducing the downregulation of the endogenous gene.

Regulatory Sequences:

A recombinant DNA construct of the present disclosure may comprise at least one regulatory sequence. A regulatory sequence may be a promoter.

A number of promoters can be used in recombinant DNA constructs of the present disclosure. The promoters can be selected based on the desired outcome, and may include constitutive, tissue-specific, inducible or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga, et al., (1999) Nature Biotechnol. 17:287-91).

Suitable constitutive promoters for use in a plant host cell include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142 and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant relevant to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful in the disclosure include soybean Kunitz trypsin inhibitor (Kti3, Jofuku and Goldberg, (1989) Plant Cell 1:1079-1093), patatin (potato tubers) (Rocha-Sosa, et al., (1989) EMBO J. 8:23-29), convicilin, vicilin, and legumin (pea cotyledons) (Rerie, et al., (1991) Mol. Gen. Genet. 259:149-157; Newbigin, et al., (1990) Planta 180:461-470; Higgins, et al., (1988) Plant. Mol. Biol. 11:683-695), zein (maize endosperm) (Schemthaner, et al., (1988) EMBO J. 7:1249-1255), phaseolin (bean cotyledon) (Segupta-Gopalan, et al., (1985) Proc. Natl. Acad. Sci. U.S.A. 82:3320-3324), phytohemagglutinin (bean cotyledon) (Voelker, et al., (1987) EMBO J. 6:3571-3577), B-conglycinin and glycinin (soybean cotyledon) (Chen, et al., (1988) EMBO J. 7:297-302), glutelin (rice endosperm), hordein (barley endosperm) (Marris, et al., (1988) Plant Mol. Biol. 10:359-366), glutenin and gliadin (wheat endosperm) (Colot, et al., (1987) EMBO J. 6:3559-3564) and sporamin (sweet potato tuberous root) (Hattori, et al., (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove, et al., (1989) Bio/Technology 7:L929-932), bean lectin and bean beta-phaseolin promoters to express luciferase (Riggs, et al., (1989) Plant Sci. 63:47-57) and wheat glutenin promoters to express chloramphenicol acetyl transferase (Colot, et al., (1987) EMBO J 6:3559-3564).

Inducible promoters selectively express an operably linked DNA sequence in response to the presence of an endogenous or exogenous stimulus, for example by chemical compounds (chemical inducers) or in response to environmental, hormonal, chemical and/or developmental signals. Inducible or regulated promoters include, for example, promoters regulated by light, heat, stress, flooding or drought, phytohormones, wounding or chemicals such as ethanol, jasmonate, salicylic acid or safeners.

Promoters for use in the current disclosure include the following: 1) the stress-inducible RD29A promoter (Kasuga, et al., (1999) Nature Biotechnol. 17:287-91); 2) the barley promoter, B22E; expression of B22E is specific to the pedicel in developing maize kernels (Klemsdal, et al., (1991) Mol. Gen. Genet. 228(1/2):9-16) and 3) maize promoter, Zag2 (Schmidt, et al., (1993) Plant Cell 5(7):729-737; Theissen, et al., (1995) Gene 156(2):155-166; NCBI GenBank Accession Number X80206)). Zag2 transcripts can be detected 5 days prior to pollination to 7 to 8 days after pollination (“DAP”), and directs expression in the carpel of developing female inflorescences and CimI which is specific to the nucleus of developing maize kernels. CimI transcript is detected 4 to 5 days before pollination to 6 to 8 DAP. Other useful promoters include any promoter which can be derived from a gene whose expression is maternally associated with developing female florets.

Additional promoters for regulating the expression of the nucleotide sequences of the present disclosure in plants are stalk-specific promoters. Such stalk-specific promoters include the alfalfa S2A promoter (GenBank Accession Number EF030816; Abrahams, et al., (1995) Plant Mol. Biol. 27:513-528) and S2B promoter (GenBank Accession Number EF030817) and the like, herein incorporated by reference.

Promoters may be derived in their entirety from a native gene or be composed of different elements derived from different promoters found in nature or even comprise synthetic DNA segments.

Promoters for use in the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank Accession Number EF030816) and S2B (Genbank Accession Number EF030817) and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO 2005/063998, published Jul. 14, 2005), the CR1BIO promoter (WO 2006/055487, published May 26, 2006), the CRWAQ81 (WO 2005/035770, published Apr. 21, 2005) and the maize ZRP2.47 promoter (NCBI Accession Number: U38790; GI Number 1063664).

Recombinant DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, (1988) Mol. Cell Biol. 8:4395-4405; Callis, et al., (1987) Genes Dev. 1:1183-1200.

Compositions:

A composition of the present disclosure is a plant comprising in its genome any of the recombinant DNA constructs of the present disclosure (such as any of the constructs discussed above). Compositions also include any progeny of the plant, and any seed obtained from the plant or its progeny, wherein the progeny or seed comprises within its genome the recombinant DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct. These seeds can be grown to produce plants that would exhibit an altered agronomic characteristic (e.g., an increased agronomic characteristic optionally under water limiting conditions) or used in a breeding program to produce hybrid seed, which can be grown to produce plants that would exhibit such an altered agronomic characteristic. The seeds may be maize seeds.

In any of the foregoing embodiments or any other embodiments of the present disclosure, the at least one agronomic characteristic may be selected from the group consisting of greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress. For example, the alteration of at least one agronomic characteristic may be an increase in yield, greenness or biomass.

“Drought” refers to a decrease in water availability to a plant that, especially when prolonged, can cause damage to the plant or prevent its successful growth (e.g., limiting plant growth or seed yield).

“Drought tolerance” is a trait of a plant to survive under drought conditions over prolonged periods of time without exhibiting substantial physiological or physical deterioration.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct in its genome exhibits increased drought tolerance relative to a reference or control plant, the reference or control plant does not comprise in its genome the recombinant DNA construct.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

The variable “% area chg_start chronic—acute2” is a measure of the percent change in total area determined by remote visible spectrum imaging between the first day of chronic stress and the day of the second acute stress

The variable “% area chg_start chronic—end chronic” is a measure of the percent change in total area determined by remote visible spectrum imaging between the first day of chronic stress and the last day of chronic stress.

The variable “% area chg_start chronic—harvest” is a measure of the percent change in total area determined by remote visible spectrum imaging between the first day of chronic stress and the day of harvest.

The variable “% area chg_start chronic—recovery24 hr” is a measure of the percent change in total area determined by remote visible spectrum imaging between the first day of chronic stress and 24 hrs into the recovery (24 hrs after acute stress 2).

The variable “psii_acute1” is a measure of Photosystem II (PSII) efficiency at the end of the first acute stress period. It provides an estimate of the efficiency at which light is absorbed by PSII antennae and is directly related to carbon dioxide assimilation within the leaf.

The variable “psii_acute2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute1” is a measure of the optimum quantum yield (Fv/Fm) at the end of the first acute stress—(variable fluorescence difference between the maximum and minimum fluorescence/maximum fluorescence).

The variable “fv/fm_acute2” is a measure of the optimum quantum yield (Fv/Fm) at the end of the second acute stress—(variable flourescence difference between the maximum and minimum fluorescence/maximum fluorescence).

The variable “leaf rolling_harvest” is a measure of the ratio of top image to side image on the day of harvest.

The variable “leaf rolling_recovery24 hr” is a measure of the ratio of top image to side image 24 hours into the recovery.

The variable “Specific Growth Rate (SGR)” represents the change in total plant surface area (as measured by an imaging instrument) over a single day (Y(t)=Y0*e^{r*t}). Y(t)=Y0*e^{r*t} is equivalent to % change in Y/Δt where the individual terms are as follows: Y(t)=Total surface area at t; Y0=Initial total surface area (estimated); r=Specific Growth Rate day⁻¹, and t=Days After Planting (“DAP”).

The variable “shoot dry weight” is a measure of the shoot weight 96 hours after being placed into a 104° C. oven.

The variable “shoot fresh weight” is a measure of the shoot weight immediately after being cut from the plant.

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% yield) in field testing under simulated or naturally-occurring drought conditions (e.g., by measuring for substantially equivalent yield under drought conditions compared to non-drought conditions or by measuring for less yield loss under drought conditions compared to a control or reference plant).

One of ordinary skill in the art would readily recognize a suitable control or reference plant to be utilized when assessing or measuring an agronomic characteristic or phenotype of a transgenic plant in any embodiment of the present disclosure in which a control plant is utilized (e.g., compositions or methods as described herein). For example, by way of non-limiting illustrations:

1. Progeny of a transformed plant which is hemizygous with respect to a recombinant DNA construct, such that the progeny are segregating into plants either comprising or not comprising the recombinant DNA construct: the progeny comprising the recombinant DNA construct would be typically measured relative to the progeny not comprising the recombinant DNA construct (i.e., the progeny not comprising the recombinant DNA construct is the control or reference plant).

2. Introgression of a recombinant DNA construct into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines and the second hybrid line is produced from the same two parent inbred lines except that one of the parent inbred lines contains a recombinant DNA construct: the second hybrid line would typically be measured relative to the first hybrid line (i.e., the first hybrid line is the control or reference plant).

4. A plant comprising a recombinant DNA construct: the plant may be assessed or measured relative to a control plant not comprising the recombinant DNA construct but otherwise having a comparable genetic background to the plant (e.g., sharing at least 90%, 91%, 92%, 93%, 94%, 95%, 96%, 97%, 98%, 99% or 100% sequence identity of nuclear genetic material compared to the plant comprising the recombinant DNA construct. There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s) and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Transgenic plants comprising or derived from plant cells of this disclosure can be further enhanced with stacked traits, e.g. a crop plant having an enhanced trait resulting from expression of DNA disclosed herein in combination with herbicide tolerance and/or pest resistance traits. For example, plants with reduced gene expression can be stacked with other traits of agronomic interest, such as a trait providing herbicide resistance and/or insect resistance, such as using a gene from Bacillus thuringensis to provide resistance against one or more of lepidopteran, coliopteran, homopteran, hemiopteran and other insects. Known genes that confer tolerance to herbicides such as e.g., auxin, HPPD, glyphosate, dicamba, glufosinate, sulfonylurea, bromoxynil and norflurazon herbicides can be stacked either as a molecular stack or a breeding stack with plants expressing the traits disclosed herein. Polynucleotide molecules encoding proteins involved in herbicide tolerance include, but are not limited to, a polynucleotide molecule encoding 5-enolpyruvylshikimate-3-phosphate synthase (EPSPS) disclosed in U.S. Pat. Nos. 39,247; 6,566,587 and for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in U.S. Pat. Nos. 7,622,641; 7,462,481; 7,531,339; 7,527,955; 7,709,709; 7,714,188 and 7,666,643 also for providing glyphosate tolerance; dicamba monooxygenase disclosed in U.S. Pat. No. 7,022,896 and WO 2007/146706 A2 for providing dicamba tolerance; a polynucleotide molecule encoding AAD12 disclosed in US Patent Application Publication Number 2005/731044 or WO 2007/053482 A2 or encoding AAD1 disclosed in US 2011/0124503 A1 or U.S. Pat. No. 7,838,733 for providing tolerance to auxin herbicides (2,4-D); a polynucleotide molecule encoding hydroxyphenylpyruvate dioxygenase (HPPD) for providing tolerance to HPPD inhibitors (e.g., hydroxyphenylpyruvate dioxygenase) disclosed in e.g., U.S. Pat. No. 7,935,869; US Patent Application Publication Number 2009/0055976 A1 and US Patent Application Publication Number 2011/0023180 A1, each publication is herein incorporated by reference in its entirety.

Other examples of herbicide-tolerance traits that could be combined with the traits disclosed herein include those conferred by polynucleotides encoding an exogenous phosphinothricin acetyltransferase, as described in U.S. Pat. Nos. 5,969,213; 5,489,520; 5,550,318; 5,874,265; 5,919,675; 5,561,236; 5,648,477; 5,646,024; 6,177,616 and 5,879,903. Plants containing an exogenous phosphinothricin acetyltransferase can exhibit improved tolerance to glufosinate herbicides, which inhibit the enzyme glutamine synthase. Other examples of herbicide-tolerance traits include those conferred by polynucleotides conferring altered protoporphyrinogen oxidase (protox) activity, as described in U.S. Pat. Nos. 6,288,306 B1; 6,282,837 B1 and 5,767,373 and International Patent Publication WO 2001/12825. Plants containing such polynucleotides can exhibit improved tolerance to any of a variety of herbicides which target the protox enzyme (also referred to as “protox inhibitors”).

The introduction of recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment or Agrobacterium-mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### EXAMPLES

The Examples described below form part of the detailed description of the disclosure. The present disclosure is further illustrated in the following Examples, in which parts and percentages are by weight and degrees are Celsius, unless otherwise stated. It should be understood that these Examples, while indicating preferred embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Frost Tolerance Screening in Maize

A. Seedling Assay:

This frost tolerance assay scores for survival at the seedling level after a freezing treatment scheme. Because this assay is done at the seedling level, high-throughput is obtained. The seedling level frost tolerance is predictive of frost tolerance at the whole plant level and through the reproductive stages of the plant, such as for example, during the grain filling stress. In an embodiment, transgenic and null seeds are planted in 4″ pot as a matched pair in greenhouse. Transformed lines from the same construct are randomized across 10 flats with 15 pots in each flat. Completely randomized block design is used to block transgenic and null plants at pot and flat level. Seedlings are grown to about V3 stage and then transferred to a growth chamber for cold acclimation at about 10° C. for 5 hours with light and at about 4° C. for 16 hours without light. After cold acclimation, the seedlings are subjected to a freezing treatment at −3° C. for up to 5.5 hours based on the transformation genotype. After freezing treatment, the seedlings are scored for survival following a 3-4 day recovery period at normal room temperature. A binary logistic regression model that uses either “1” for survival or “0” for a dead plant provides logarithm of probability ratio of survived/dead. The null hypothesis is transgenic plants have the same survival as the controls. If the transgenic plants have higher survival than controls at either the 0.05 or 0.1 level, then the null hypothesis is rejected.

B. Reproductive Plant Assay:

This frost tolerance assay is performed at the reproductive stages of the plant (e.g., corn plants). Transgenic and null seeds are planted in 4″ pot in greenhouse. After the seedlings reached to V3 they were transplanted to 9″ 1 gallon pot until about R3-4 stage. For cold acclimation, approximately 10 transgenic and 10 null plants were subjected to 10° C. for 5 hours and 4° C. for 16 hours. After cold acclimation, the transgenic and null plants were moved to cold chamber to undergo freezing treatment. During the −3° C., 1 hour treatment, nulls/controls are placed between transgenic plant to reduce position effect. Plants are allowed to recover at room temperature with chlorophyll fluorescence measured at 1, 5 and 24 hours after the freezing treatment. Higher chlorophyll florescence indicates a higher tolerance to freezing.

### Example 2

### Engineering Frost Tolerance in Maize Using a Kinase

Nicotiana Protein Kinase1 (NPK1) is a mitogen activated protein kinase kinase kinase that is involved in cytokinesis regulation and oxidative stress signal transduction. The ZmNPK1B which has about 70% amino acid similarity to rice NPKL3 was tested for frost tolerance in maize seedlings and reproductive stages. In the seedling assay described in Example 1, approximately 2900 plants were tested for survival. Six out of nine events showed that transgenic seedlings had significantly higher survival than control (Table 1). The transgenic had significant higher survival % than null on construct level as well. For reproductive stage frost tolerance, chlorophyll fluorescence of 20 plants from the line 1.23 was measured at 1 hour, 5 hours and 24 hours during recovery due to resource limitation. Significant higher chlorophyll fluorescence value for transgenic plants than nulls was observed (see, Table 2). Thus, the seedling assay for the transgenic construct is correlated with the reproductive frost tolerance assay.

The gene expression data for NPK1 from seedlings is in Table 3. Each data point is the average value from 3 seedling samples. An inducible promoter Rab17 was used. No gene expression was detected from null plants across all treatments. The gene seemed inducted after cold acclimation and during −3° C. treatment period in most of the events but at low levels.

TABLE 1
Seedling Survival of RAB17::ZM-NPK1B at −3 C.

| | 10 Experiments | | | | |
|---|---|---|---|---|---|
| | Transgene + | Control | S % | | P |
| Line | S %* | S % | Diff | Rep# | value |
| 1.22 | 60 | 45.1 | 14.9 | 165 | 0.0144 |
| 1.23 | 73.5 | 52.2 | 21.3 | 163 | 0.0004 |
| 2.13 | 64.3 | 55.3 | 9 | 157 | 0.142 |
| 2.23 | 60 | 43.4 | 16.6 | 156 | 0.0131 |
| 2.34 | 61.4 | 53.9 | 7.5 | 160 | 0.2127 |
| 2.37 | 63 | 51.3 | 11.7 | 163 | 0.0554 |
| 2.40 | 63.1 | 52.5 | 10.6 | 162 | 0.0849 |
| 2.41 | 62.4 | 54.8 | 7.6 | 168 | 0.1996 |
| 2.49 | 61.1 | 45.8 | 15.3 | 162 | 0.0143 |
| Construct | 63.3 | 50.5 | 12.8 | 1456 | <.0001 |

Control—Null & WT; freezing duration from 3 to 5.5 h
*S % = % survival;
P < 0.1

TABLE 2
Chlorophyll Fluorescence of 1.23

| Recovery | | | | | |
|---|---|---|---|---|---|
| Hour After | | | | | |
| Freezing | Transgene | φPSII | StdErr | DF | tValue |
| 0 | Neg | 0.555 | 0.014 | 16 | 40.55 |
| 0 | Pos | 0.556 | 0.014 | 16 | 40.6 |
| 1 | Neg | 0.497 | 0.019 | 20 | 26.13 |
| 1 | Pos | 0.483 | 0.019 | 20 | 25.41 |
| 5 | Neg | 0.520 | 0.016 | 15 | 32.59 |
| 5 | Pos | 0.536 | 0.016 | 15 | 33.59 |
| 24 | Neg | 0.430 | 0.036 | 20 | 11.92 |
| 24 | Pos | 0.530 | 0.036 | 20 | 14.69 |

TABLE 3
RAB17::ZM-NPK1B Gene Expression in Seedlings

| | | Relative Gene |
|---|---|---|
| Line # | Treatment | Expression |
| Null | before cold acclimation | 0.0000000 |
| 1.22 | before cold acclimation | 0.0006381 |
| 1.23 | before cold acclimation | 0.0000000 |
| 2.13 | before cold acclimation | 0.0008877 |
| 2.37 | before cold acclimation | 0.0017102 |
| 2.40 | before cold acclimation | 0.0011702 |
| 2.41 | before cold acclimation | 0.0000000 |
| 2.49 | before cold acclimation | 0.0005189 |
| Null | after cold acclimation | 0.0000000 |
| 1.22 | after cold acclimation | 0.0019337 |
| 1.23 | after cold acclimation | 0.0015620 |
| 2.13 | after cold acclimation | 0.0019403 |
| 2.23 | after cold acclimation | 0.0000079 |
| 2.34 | after cold acclimation | 0.0000000 |
| 2.37 | after cold acclimation | 0.0119787 |
| 2.40 | after cold acclimation | 0.0004138 |
| 2.41 | after cold acclimation | 0.0011547 |
| 2.49 | after cold acclimation | 0.0000000 |
| Null | −3 C. for 1 hour | 0.0000000 |
| 1.22 | −3 C. for 1 hour | 0.0023245 |
| 1.23 | −3 C. for 1 hour | 0.0018401 |
| 2.13 | −3 C. for 1 hour | 0.0032720 |
| 2.23 | −3 C. for 1 hour | 0.0000000 |
| 2.34 | −3 C. for 1 hour | 0.0000000 |
| 2.37 | −3 C. for 1 hour | 0.0188456 |
| 2.40 | −3 C. for 1 hour | 0.0000000 |
| 2.41 | −3 C. for 1 hour | 0.0021763 |
| 2.49 | −3 C. for 1 hour | 0.0019813 |

### Example 3

### Engineering Frost Tolerance in Maize Using a Transcription Factor

TaDREB3 is a Dehydration Responsive Element Binding Protein from wheat. Its gene product is an AP2-domain DNA binding transcription factor involved in abiotic stress signal transduction. In seedling assay, approximately 1800 plants were tested for survival. Three out of ten events showed that transgenic seedlings had significantly higher survival than control. The transgenic had significant higher survival % than null on construct level as well. (see, Table 4).

The gene expression data from seedlings is shown in Table 5. Each data point is the average value from 3 seedling samples. No gene expression was detected from null plants across all treatments. The gene was induced after cold acclimation at 4° C. and during −3° C. treatment from 2 to 4 hours in most of the events.

TABLE 4
Survival of ZMLIP15::TA-DREB3 at −3 C.

| | 6 Experiments | | | | |
|---|---|---|---|---|---|
| | Transgene + | Control | S Diff | | |
| Line # | S % | S % | % | Rep# | P value |
| 5.2.12 | 36.6 | 27.6 | 9 | 85 | 0.233 |
| 5.3.1 | 48.6 | 43.1 | 5.5 | 82 | 0.5109 |
| 5.3.11 | 43.9 | 40.5 | 3.4 | 95 | 0.6614 |
| 5.3.2 | 37.4 | 47.5 | −10.1 | 92 | 0.2457 |
| 5.3.3 | 12.9 | 5.9 | 7 | 87 | 0.0328 |
| 5.3.6 | 56.8 | 53.8 | 3 | 91 | 0.7463 |
| 5.5.1 | 67.3 | 39.4 | 27.9 | 88 | 0.0031 |
| 5.5.7 | 45.3 | 30.3 | 15 | 93 | 0.0446 |
| 5.6.5 | 34 | 35 | −1 | 94 | 0.9003 |
| 5.6.8 | 45.8 | 38.5 | 7.3 | 89 | 0.3794 |
| Construct | 41.8 | 33.9 | 7.9 | 896 | 0.0024 |

Control—Null & WT; freezing duration 3-5 hours.

TABLE 5
RAB17::ZM-NPK1B Gene Expression in Seedlings

| | | Relative Gene |
|---|---|---|
| Line # | Treatment | Expression |
| Null | before cold acclimation | 0.0000 |
| 5.2.12 | before cold acclimation | 0.0306 |
| 5.3.1 | before cold acclimation | 0.0151 |
| 5.3.11 | before cold acclimation | 0.0135 |
| 5.3.2 | before cold acclimation | 0.0220 |
| 5.3.3 | before cold acclimation | 0.0395 |
| 5.3.6 | before cold acclimation | 0.0260 |
| 5.5.1 | before cold acclimation | 0.0314 |
| 5.5.7 | before cold acclimation | 0.0000 |
| 5.6.5 | before cold acclimation | 0.0258 |
| 5.6.8 | before cold acclimation | 0.0116 |
| Null | after cold acclimation | 0.0000 |
| 5.2.12 | after cold acclimation | 0.1146 |
| 5.3.1 | after cold acclimation | 0.1927 |
| 5.3.11 | after cold acclimation | 0.1179 |
| 5.3.2 | after cold acclimation | 0.1934 |
| 5.3.3 | after cold acclimation | 0.3158 |
| 5.3.6 | after cold acclimation | 0.0775 |
| 5.5.1 | after cold acclimation | 0.0767 |
| 5.5.7 | after cold acclimation | 0.0000 |
| 5.6.5 | after cold acclimation | 0.0877 |
| 5.6.8 | after cold acclimation | 0.0519 |
| Null | −3 C. for 2 hours | 0.0000 |
| 5.2.12 | −3 C. for 2 hours | 0.0477 |
| 5.3.1 | −3 C. for 2 hours | 0.0765 |
| 5.3.11 | −3 C. for 2 hours | 0.0382 |
| 5.3.2 | −3 C. for 2 hours | 0.0663 |
| 5.3.3 | −3 C. for 2 hours | 0.1684 |
| 5.3.6 | −3 C. for 2 hours | 0.0467 |
| 5.5.1 | −3 C. for 2 hours | 0.0502 |
| 5.5.7 | −3 C. for 2 hours | 0.0000 |
| 5.6.5 | −3 C. for 2 hours | 0.0620 |
| 5.6.8 | −3 C. for 2 hours | 0.0739 |
| Null | −3 C. for 4 hours | 0.0000 |
| 5.2.12 | −3 C. for 4 hours | 0.0624 |
| 5.3.1 | −3 C. for 4 hours | 0.1040 |
| 5.3.11 | −3 C. for 4 hours | 0.0797 |
| 5.3.2 | −3 C. for 4 hours | 0.0971 |
| 5.3.3 | −3 C. for 4 hours | 0.1240 |
| 5.3.6 | −3 C. for 4 hours | 0.0631 |
| 5.5.1 | −3 C. for 4 hours | 0.0772 |
| 5.5.7 | −3 C. for 4 hours | 0.0000 |
| 5.6.5 | −3 C. for 4 hours | 0.0860 |
| 5.6.8 | −3 C. for 4 hours | 0.0641 |

In summary, the NPK1 expressing maize transgenic seedlings showed significant frost tolerance phenotype.

### Example 4

### Shortening Maturity Via Manipulation of Early Flowering Phenotype with FTM1 Expression

The purpose of this experiment was to demonstrate that overall plant maturity could be shortened by modulating the flowering time phenotype of plants through expressing a transgene. Such a phenotype modification can also be achieved with additional transgenes or through a breeding approach.

FTM1 stands for Floral Transition MADS 1 transcription factor. It is a MADS Box transcriptional factor and induces floral transition. As demonstrated herein, the transgenic phenotype upon over-expression is early flowering.

Upon expression under constitutive promoter, the transgenic plants exhibited early flowering and shortened maturity, but surprisingly ear and tassel developed normally as compared to the wild-type plants. In addition, the plants had reduced plant height and reduced leaf size. The inbred yield vigor was low, but the yield vigor in the hybrid background was relatively higher.

TABLE 6A
Maturity and morphology traits affected
by UBI::FTM1 in top-cross hybrid.

| | | | | Plant | Ear |
|---|---|---|---|---|---|
| | | | MST | Height | Height |
| Event | GDUSHD | GDUSLK | (%) | (in) | (in) |
| Wildtype | 1366.1 | 1420.3 | 20.76 | 104.52 | 39.42 |
| EVENTS (5) | 1228.58 | 1291.04 | 17.68 | 84.65 | 24.01 |
| Difference | −137.52 | −129.26 | −3.08 | −19.87 | −15.41 |
| % Change | −10.1% | −9.1% | −14.9% | −19.0% | −39.1% |

Data shown are average values across locations and event/plant replications, from field planting.
GDUSHD—accumulative GDU to shedding; GDUSLK—accumulative GDU to silking; MST (%)—percent grain moisture at harvest.

TABLE 6B
Maturity reduction in UBI:FTM1 hybrids

| | | Maturity with FTM1 |
|---|---|---|
| Genotype | Maturity | transgene |
| UBI::FTM1 transgenics | Not determined | 7-10 days earlier flowering |
| UBI::FTM1 null | 119 | |
| Inbred tester 1 | 92 | |
| Inbred tester 2 | 92 | |
| Tester 1 hybrid | 103 | 7 days earlier flowering |
| Tester 2 hybrid | 110 | 4 days earlier flowering |

Individual trait measurements shown in Table 6A are commonly associated with maturity. GDUSHD and GDUSLK reflect thermal time for plant to reach anthesis. MST is the primary measurement of grain dry-down process and impacts yield directly. As the transgenic plants flowers earlier than the wildtype, ear and plant heights are lowered, consistent with the flowering time modification. Table 6B demonstrates the reduction in relative maturity of FTM1 expressing transgenic maize plants with different inbred testers.

Following the above mentioned field testing in Table 6B, additional hybrid material was created using short-season germplasm native to northern locations carrying the UBI::FTM1 transgene. This material was tested in several northern dry climatic regions locations, potential target environments for this adapted hybrid, under normal nitrogen levels (about 150 lbs/acre) for the tested locations. The transgenic plants showed an average of 30 GDU earlier in time to flowering, and 5 points reduction in grain moisture (MST). Average yield was measured to be about 110 bu/acre for the transgenics, compared to about 125 bu/acre for the wild-type. This approximately translates to an equivalent of 5 CRM reduction in maturity rating. These results demonstrate that the FTM1 gene was utilized in creating hybrid materials with shortened maturity in short-season environments.

In summary, FTM1-expressing maize plants demonstrated that by manipulating a floral transition gene, time to flowering can be reduced significantly, leading to a shortened maturity for the plant. As maturity can be generally described as time from seeding to harvest, a shorter maturity is relevant for ensuring that a crop can finish in the northern continental dry climatic environment.

### Example 5

### Shortening Maturity Via Manipulation of Early Flowering Phenotype with ZmRap2.7 Down-Regulation

This experiment was performed to demonstrate that overall plant maturity could be shortened by modulating the flowering time phenotype of plants through modulation by a transgene. Shortening of plant maturity was obtained by an early flowering phenotype.

RAP2.7 is an acronym for Related to APETALA 2.7. RAPL means RAP2.7 LIKE and RAP2.7 functions as an AP2-family transcription factor that suppresses floral transition. It may also be regulated by a miRNA miR172 target. Transgenic phenotype upon silencing or knock-down of Rap2.7 resulted in early flowering, reduced plant height, but surprisingly developed normal ear and tassel as compared the wild-type plants. Overexpression of Rap2.7 resulted in delayed flowering and larger plant size, confirming that Rap2.7 is a negative regulatory of gene expression.

TABLE 7
(A-B) Maturity and morphology traits affected
by ACTIN::RAP2.7 RNAi in top-cross hybrid.
(A) Year 1, Location 1 data - 2 events

| | | | Plant | Ear |
|---|---|---|---|---|
| | | | Height | Height |
| Event | GDUSHD | GDUSLK | (in) | (in) |
| Wildtype | 1260 | 1270 | 111 | 51 |
| EVENTS(2) | 1130 | 1145 | 102 | 37.5 |
| Difference | −130 | −125 | −9 | −13.5 |
| % Change | −10.3% | −9.8% | −8.1% | −26.5% |

Data shown are average values across event/plant replications, from field
planting.

(B) Year 2, Location 2 data - 1 event

| | | | MST |
|---|---|---|---|
| Event | GDUSHD | GDUSLK | (%) |
| Wildtype | 1331 | 1324 | 23 |
| EVENTS(2) | 1179 | 1213 | 20 |
| Difference | −152 | −111 | −3 |
| % Change | −11.4% | −8.4% | −13.0% |

GDUSHD—accumulative GDU to shedding; GDUSLK—accumulative
GDU to silking; MST (%)—percent grain moisture at harvest.

Individual trait measurements shown in Table 7 above are commonly associated with maturity. GDUSHD and GDUSLK reflect thermal time for plant to reach anthesis. MST is the primary measurement of grain dry-down process, and impacts yield directly. As the transgenic plants flowers earlier than the wildtype, ear and plant heights are lowered.

Allelic Diversity of RAP2.7 Gene in Maize Germplasm

Significant sequence variations exist for RAP2.7 gene in corn. Such variations include haplotypes of multiple SNPs and insertion/deletions as large as 60 nucleotides. These variations will need to be taken into consideration for efficacy of gene silencing depending on the germplasm.

The sequence polymorphisms observed for RAP2.7 alleles can potentially mean functional diversity. For example, germplasm variations for Rap2.7 can be exploited to reduced flowering time through marker assisted selection of early flowering alleles. When correlations are established between specific alleles and flowering time phenotype, molecular markers can be developed for selection in breeding towards flowering time changes, either early up or extend maturity of a given inbred. Genetic variations for early flowering time can thus be engineered to shorten plant maturity in combination with a transgenic or a breeding approach.

### Example 6

### Early Flowering Phenotype Due to Stacking of FTM1 and Rap2.7

Transgenic plants carrying either UBI::FTM1 or ACTIN::RAP2.7 RNAi constructs have been established to promote early flowering. When these plants were crossed, F1 progeny flowered earlier than either parent, indicating that the transgene effect from FTM1 and RAP2.7 can be stacked and further shorten the time to flowering. Leaf numbers are used to here to show the earliness of flowering, as earlier floral transition results in fewer leaves overall. The ear and plant height data provide further support for the early-flowering phenotype since early-flowering plants are shorter. The stay-green scores are arbitrary ratings of plant senescence towards the end of season, with lower scores reflecting more advanced stages of senescence for the plant. Early senescence is generally desirable for faster dry down of grains towards the end of a growing season. It is relevant to faster dry down in a growing season that is generally short for example, in the northern dry climatic regions of interest.

A prolonged stay-green or poor dry down usually leads to crops standing late into the fall or early winter since farmers are unable to harvest the grains with high moisture. This inevitably results in yield loss. Having a faster dry down is relevant for the northern continental dry climatic regions due to the short frost free period.

TABLE 8
Breeding stack between FTM1 and RAP2.7 transgenic plants

| | | Plant | Ear | |
|---|---|---|---|---|
| | Leaf | Height | Height | Stay |
| Construct | Number | (in) | (in) | Green |
| Wild type | 18.1 | 100 | 45 | 9.0 |
| FTM1 | 13.5 | 93 | 31 | 5.5 |
| RAP2.7 | 13.7 | 86.5 | 28 | 8.5 |
| Breeding Stack of | 11.7 | 81 | 22.3 | 6.7 |
| FTM1 × Rap2.7 | | | | |

Transgenic plants carrying either UBI::FTM1 or ACTIN::RAP2.7 RNAi constructs have been established to promote early flowering. When these plants were crossed, F1 progeny flowered earlier than either parent, indicating that the transgene effect from FTM1 and RAP2.7 can be stacked and further shorten the time to flowering. Leaf numbers are used to here to show the earliness of flowering, as earlier floral transition results in fewer leaves overall. The ear and plant height data provide further support for the early-flowering phenotype since early-flowering plants are shorter. The stay-green scores are arbitrary ratings of plant senescence towards the end of season, with lower scores reflecting more senescence of the plant.

### Example 7

### Engineering Architecture Modification for Maize

The purpose of this experiment was to demonstrate architecture modification to further enable adapting corn to grow in the northern dry climatic regions of interest. Agronomic augmentation for root and stalk lodging improvement by a variety of genes are described in this Example. In conjunction with the shortening maturity constructs, the construct containing cellulose synthase A4 was used for architecture modification.

Construct 37407—F3.7::CesA4+FTM1::DD+NAS2::DD+S2A::D8mpl+35S::BAR)

The Intended phenotype are as follows:

F3.7::CesA4—stronger stalks; F3.7 is a maize stalk-preferred promoter

FTM1::DD+NAS2::DD—increase elongation in tassel and root; NAS2 is a maize root-preferred promoter

S2A::D8mpl-Stature Reduction; S2A is an alfalfa stalk-preferred promoter; D8mpl encodes a truncated form of maize Dwarf 8 gene.

Selective organ architecture modification was achieved through manipulation of the D8 dimerization domain (DD). Manipulation of plant architecture is described for example in US Patent Application Publication Number 2011/0023190, incorporated herein by reference to the extent it relates to the use of dimerization domain for modifying plant architecture.

TABLE 9
Architecture modification of transgenic plants.

| | | Plant | |
|---|---|---|---|
| | | Height | Height |
| Background | Genotype | (in) | Reduction |
| EF247TX/Tester1 | 37407 | 76 | 31% |
| | Wildtype | 109 | |
| EF247TX/Tester2 | 37407 | 81 | 27% |
| | Wildtype | 112 | |
| EF247TX/Tester3 | 37407 | 74 | 32% |
| | Wildtype | 109 | |
| EF247TX/Tester4 | 37407 | 68 | 36% |
| | Wildtype | 105 | |

Plant height was reduced for PHP37407 across 4 testers in hybrid background, data collected from plantings in Year 1, Location 1.

The dwarfing stack construct 37407 has consistently resulted in plant height reduction that averaged 30% across 4 testers in top-cross hybrids. The transgenic plants had healthy canopy and produced ears that were comparable in size as those produced by wild-type plants.

In summary, as shown in Table 9, the dwarfing construct resulted in a moderated dwarfing phenotype where overall plant height has been reduced by an average of 30% regardless of the tester inbred used. These plants were ideal materials for agronomic practices such as higher planting densities to increase yield on a per land area basis, without the high risk of lodging that is normally associated with high planting densities (see below).

TABLE 10
Root lodging % reduction by 37407.

| Event | 32,000 | 40,000 | 48,000 | Average |
|---|---|---|---|---|
| 84.1.17 | 9 | 7 | 10 | 9 |
| 84.1.3 | 9 | 6 | 10 | 8 |
| 84.1.4 | 9 | 6 | 10 | 8 |
| 84.2.3 | 9 | 7 | 10 | 8 |
| 84.2.5 | 9 | 7 | 10 | 8 |
| 84.2.6 | 9 | 8 | 10 | 9 |
| 84.3.11 | 9 | 6 | 10 | 8 |
| 84.3.4 | 9 | 6 | 10 | 9 |
| 84.3.8 | 9 | 6 | 10 | 8 |
| 84.4.3 | 9 | 7 | 10 | 8 |
| 37407 (construct average) | 9 | 6 | 10 | 8 |
| Bulked nulls | 24 | 26 | 35 | 28 |

Root lodging was reduced in 37407 across 3 planting densities in hybrid background, data shown are percentage of plants that were root lodged, collected from plantings in Year 1, Location 1. (See, Table 10) In Year 1, rain storms led to root lodging that affected plantings in Location 1. However, all 10 events from construct 37407 were observed to have a consistent 20% less root lodging compared to the non-transgenic null plants, with the transgenic plants averaging 8% lodged versus the nulls with 28% lodged, across all 3 planting densities—32,000, 40,000 and 48,000 plants per acre.

TABLE 11
Root lodging across different planting
densities for construct 37407.

| Event | 32,000 | 40,000 | 48,000 | All |
|---|---|---|---|---|
| 84.1.17 | 0 | 0 | 0 | 0 |
| 84.1.3 | 0 | 0 | 0 | 0 |
| 84.1.4 | 0 | 0 | 0 | 0 |
| 84.2.3 | 0 | 0 | 0 | 0 |
| 84.2.5 | 0 | 0 | 0 | 0 |
| 84.2.6 | 0 | 0 | 0 | 0 |
| 84.3.11 | 0 | 0 | 0 | 0 |
| 84.3.4 | 0 | 0 | 0 | 0 |
| 84.3.8 | 0 | 0 | 0 | 0 |
| 84.4.3 | 0 | 0 | 0 | 0 |
| 37407 (construct average) | 0 | 0 | 0 | 0 |
| Bulked nulls | 11 | 7 | 2 | 6 |

Root lodging across 3 planting densities in hybrid background are shown in Table 11, data shown are percentage of plants that were root lodged, collected from plantings in Year 1, Location 1. In Year 1, testing plots in Location 2 were hit with wind storms that caused wide-spread brittle snap. All 10 events from 37407 had no plants showing brittle snap, whereas bulked nulls had an average of 6% snapped plants across all 3 planting densities—32,000, 40,000 and 48,000 plants per acre.

In summary, as shown in Tables 10-11, the construct 37407 resulted in reduced root lodging phenotype and better resistance to brittle snap. The increased root and stalk strength is essential for the utility of these dwarf materials in high planting density environment, further realizing the true potential of semi-dwarf plant type.

### Example 8

### Transformation of Maize Using Agrobacterium

Agrobacterium-mediated transformation of maize is performed for example, as described by Zhao, et al., (2006) Meth. Mol. Biol. 318:315-323 (see also, Zhao, et al., (2001) Mol. Breed. 8:323-333 and U.S. Pat. No. 5,981,840 issued Nov. 9, 1999, incorporated herein by reference). The transformation process involves bacterium innoculation, co-cultivation, resting, selection and plant regeneration.

1. Immature Embryo Preparation:

Immature maize embryos are dissected from caryopses and placed in a 2 mL microtube containing 2 mL PHI-A medium.

2. Agrobacterium Infection and Co-Cultivation of Immature Embryos:

2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is relevant for recovering stable transgenic events.

3. Selection of Putative Transgenic Events:

To each plate of PHI-D medium in a 100×15 mm Petri dish, 10 embryos are transferred, maintaining orientation and the dishes are sealed with PARAFILM®. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 Plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

Media for Plant Transformation:
1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemente with 0.85 mg/L silver nitrate (filter-sterilized), 3.0 g/L GELRITE®, 100 μM acetosyringone (filter-sterilized), pH 5.8.
3. PHI-C: PHI-B without GELRITE® and acetosyringonee, reduce 2,4-D to 1.5 mg/L and supplemente with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L GELRITE®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm, et al., (1990) Bio/Technology 8:833-839).

Transgenic T0 plants can be regenerated and their phenotype determined. T1 seed can be collected. T1 plants, and/or their progeny, can be grown and their phenotype determined.

### Example 9

### Yield Analysis of Plants Transformed with Targeting Constructs

A recombinant DNA construct containing a gene or suppression element of interest can be introduced into plants either by direct transformation or introgression from a separately transformed line.

Transgenic plants, either inbred or hybrid, can undergo more vigorous field-based experiments to study yield enhancement and/or stability under well-watered and water-limiting conditions.

Subsequent yield analysis can be done to determine whether plants that contain the constructs/sequences disclosed herein have an improvement in yield performance under water-limiting conditions, when compared to the control plants that do not contain the validated drought tolerant lead gene. Specifically, drought conditions can be imposed during the flowering and/or grain fill period for plants that contain the constructs/sequences disclosed herein and the control plants. Reduction in yield can be measured for both. Plants containing the constructs/sequences disclosed herein have less yield loss relative to the control plants, for example, at least 25% less yield loss, under water limiting conditions, or would have increased yield relative to the control plants under water non-limiting conditions.

The above method may be used to select transgenic plants with increased yield, under water-limiting conditions and/or well-watered conditions, when compared to a control plant not comprising said recombinant DNA construct.

1. A method of increasing yield by adapting corn plant to grow in a crop-growing environment characterized as northern continental dry climatic region having an average annual CHU of about 1700 to 2000 when measured in ° F. or an average annual GDU of about 1400 to about 1700 when measured in ° F., the method comprises:
a. expressing one or more recombinant nucleic acids conferring a frost tolerant phenotype when the plant is exposed to −3° C. for about 3 hours;
b. expressing one or more recombinant nucleic acids that reduce maturity of corn to about a comparative relative maturity of about 60-70 or wherein a reduction of about 4-10 days in maturity is achieved when compared to a control plant not having the recombinant nucleic acids; and
c. increasing the yield of corn to a yield of at least about 100 bu/acre.

2. The method of claim 1, wherein the corn plant further comprises a recombinant nucleic acid that increases harvest index and optionally reduces plant stature including plant height.

3. The method of claim 2, wherein the corn plant is capable of being planted at a higher population density compared to corn plants not comprising the recombinant nucleic acid.

4. The method of claim 1, wherein the corn plant is chilling tolerant after being exposed to temperatures of less than about 15° C.

5. The method of claim 1, wherein the corn plant is exposed to frost during a seedling stage.

6. The method of claim 1, wherein the corn plant is exposed to frost during grain filling stage.

7. The method of claim 1, wherein the corn plant further comprises a modified plant architecture or change in harvest index through the modulation of one or more transgenes.

8. The method of claim 7, wherein the modified plant architecture comprises a modification selected from the group consisting of increased harvest index, shorter stature, reduced leaf angle, and reduced canopy.

9. The method of claim 1, wherein the relative maturity of corn is reduced by modulating a maturity parameter selected from the group consisting of flowering time, grain filling, and senescence.

10. (canceled)

11. The method of claim 1, wherein the plants are planted at a planting density of about 20,000 plants to about 50,000 plants per acre.

12. The method of claim 1, wherein the frost tolerance phenotype is conferred by transgenic modulation of one or more nucleic acids that provide chilling or frost tolerance.

13. The method of claim 9, wherein the plant architecture is modified by transgenic modulation of one or more nucleic acids selected from the group consisting of maturity reducing genes, dwarfing genes, growth suppressing genes, moderated dwarfing genes and Della proteins or a gene involved in biosynthesis, metabolism of and response to phytohormone Gibberellic acid.

14. The method of claim 1, wherein the corn does not exhibit a negative agronomic characteristic comprising root lodging or stalk lodging due to early maturity.

15. The method of claim 1, wherein the corn further comprises a genetic modification for premature senescence.

16. A method of increasing yield by adapting corn plant to grow in a crop-growing environment characterized as northern continental dry climatic region, the method comprises:
a. expressing one or more recombinant nucleic acids conferring a frost tolerant phenotype when the corn plant is exposed to about −3° C. for about 3 hours;
b. selecting a genetic modification that reduces the maturity of corn to about a comparative relative maturity of about 60-70 or wherein a reduction of about 4-10 days in maturity is achieved in the corn plant when compared to a control corn plant not having said genetic modifications; and
c. increasing the yield of corn to an average yield of at least about 100 bu/acre.

17. The method of claim 16, wherein the genetic modification is selected through marker-assisted breeding.

18. The method of claim 16, wherein the genetic modification comprises a single nucleotide polymorphism (SNP) marker.

19. The method of claim 16, wherein the genetic modification comprises a quantitative trait locus.

20. (canceled)

21. (canceled)

22. (canceled)

23. A method of screening for corn plants that are tolerance to freezing, the method comprising
a. acclimatizing corn seedlings at about V2-V4 stage at about 8-12° C. for about 4-6 hours followed by a cold treatment at about 3-5° C. for about 14-18 hours under no light;
b. treating the acclimatized seedlings to a freezing condition of about −2° C. to −3° C. for about 3-6 hours depending on the genotype of the seedlings;
c. transferring the seedlings to room temperature; and
d. screening the seedlings for survival after 3-5 days.

24. The method of claim 20, wherein the seedling is a transgenic seedling comprising a recombinant nucleic acid.

25. (canceled)

26. The method of claim 20, wherein the screening method comprises assigning a binary value for survival or death of the seedlings.

27. The method of claim 20, wherein the cold acclimatization of the seedlings is performed in a growth chamber.

28-52. (canceled)


SEQUENCE LISTING (36 sequences)

SEQ ID NO: 1 - protein, 245 aa, Zea mays
MGRGKVQLKRIENKINRQVTFSKRRSGLLKKAHEISVLCDAEVALIIFSTKGKLYEYSTDSCMDKILERYERYSYAEKVLISAEYETQGNWCHEYRKLKAKVETIQKCQKHLMGEDLETLNLKELQQLEQQLESSLKHIRTRKSQLMVESISALQRKEKSLQEENKVLQKELAEKQKDQRQQVQRDQTQQQTSSSSTSFMLREAAPTTNVSIFPVAAGGRVVEGAAAQPQARVGLPPWMLSHLSC

SEQ ID NO: 2 - DNA, 738 bp, Zea mays
atggggcgcgggaaggtgcagctgaagcggatcgagaacaagatcaaccgccaggtgacattctccaagcgccgctcggggctactcaagaaggcgcacgagatctccgtgctctgcgacgccgaggtcgcgctcatcatcttctccaccaagggcaagctctacgagtactctaccgattcatgtatggacaaaattcttgaacggtatgagcgctactcctatgcagaaaaggttctcatttccgcagaatatgaaactcagggcaattggtgccatgaatatagaaaactaaaggcgaaggtcgagacaatacagaaatgtcaaaagcacctcatgggagaggatcttgaaactttgaatctcaaagagcttcagcaactagagcagcagctggagagttcactgaaacatatcagaacaaggaagagccagcttatggtcgagtcaatttcagcgctccaacggaaggagaagtcactgcaggaggagaacaaggttctgcagaaggagctcgcggagaagcagaaagaccagcggcagcaagtgcaacgggaccaaactcaacagcagaccagttcgtcttccacgtccttcatgttaagggaagctgccccaacaacaaatgtcagcatcttccctgtggcagcaggcgggagggtggtggaaggggcagcagcgcagccgcaggctcgcgttggactgccaccatggatgcttagccatctgagctgctga

SEQ ID NO: 3 - DNA, 1991 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 4 - DNA, 2144 bp, Oryza sativa
atccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgttttttttttccgtcacggtctcgatctttggccttggtagtttgggtgggcgagaggcggcttcgtgcgcgcccagatcggtgcgcgggaggggcgggatctcgcggctggggctctcgccggcgtggatcaggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtag

SEQ ID NO: 5 - protein, 470 aa, Zea mays
MQLDLNVAEAPPPVEMEASDSGSSVLNASEAASAGGAPAPAEEGSSSTPAVLEFSILIRSDSDAAGADEDEDATPSPPPRHRHQHQQQLVTRELFPAGAGPPAPTPRHWAELGFFRADLQQQQAPGPRIVPHPHAAPPPAKKSRRGPRSRSSQYRGVTFYRRTGRWESHIWDCGKQVYLGGFDTAHAAARAYDRAAIKFRGVDADINFNLSDYEDDMKQMGSLSKEEFVHVLRRQSTGFSRGSSRYRGVTLHKCGRWEARMGQFLGKKYIYLGLFDSEVEAARAYDKAAIKCNGREAVTNFEPSTYHGELPTEVADVDLNLSISQPSPQRDKNSCLGLQLHHGPFEGSELKKTKIDDAPSELPGRPRQLSPLVAEHPPAWPAQPPHPFFVFTNHEMSASGDLHRRPAGAVPSWAWQVAAAAPPPAALPSSAAASSGFSNTATTAATTAPSASSLRYCPPPPPPSSHHHPR

SEQ ID NO: 6 - DNA, 1413 bp, Zea mays
atgcagttggatctgaacgtggccgaggcgccgccgccggtggagatggaggcgagcgactcggggtcgtcggtgctgaacgcgtcggaagcggcgtcggcgggcggcgcgcccgcgccggcggaggagggatctagctcaacgccggccgtgctggagttcagcatcctcatccggagcgatagcgacgcggccggcgcggacgaggacgaggacgccacgccatcgcctcctcctcgccaccgccaccagcaccagcagcagctcgtgacccgcgagctgttcccggccggcgccggtccgccggccccgacgccgcggcattgggccgagctcggcttcttccgcgccgacctgcagcagcaacaggcgccgggccccaggatcgtgccgcacccacacgccgcgccgccgccggccaagaagagccgccgcggcccgcgctcccgcagctcgcagtaccgcggcgtcaccttctaccgccgcacaggccgctgggagtcccacatctgggattgcggcaagcaggtgtacctaggtggattcgacaccgctcacgccgctgcaagggcgtacgaccgggcggcgatcaagttccgcggcgtcgacgccgacatcaacttcaacctcagcgactacgaggacgacatgaagcagatggggagcctgtccaaggaggagttcgtgcacgtcctgcgccgtcagagcaccggcttctcgagaggcagctccaggtacagaggcgtcaccctgcacaagtgcggccgctgggaggcgcgcatggggcagttcctcggcaagaagtacatataccttgggctattcgacagcgaagtagaggctgcaagagcctacgacaaggccgccatcaaatgcaatggcagagaggccgtgacgaacttcgagccgagcacgtatcacggggagctgccgactgaagttgctgatgtcgatctgaacctgagcatatctcagccgagcccccaaagagacaagaacagctgcctaggtctgcagctccaccacggaccattcgagggctccgaactgaagaaaaccaagatcgacgatgctccctctgagctaccgggccgccctcgtcagctgtctcctctcgtggctgagcatccgccggcctggcctgcgcagccgcctcaccccttcttcgtcttcacaaaccatgagatgagtgcatcaggagatctccacaggaggcctgcaggggctgttcccagctgggcatggcaggtggcagcagcagctcctcctcctgccgccctgccgtcgtccgctgcagcatcatcaggattctccaacaccgccacgacagctgccaccaccgccccatcggcctcctccctccggtactgcccgccgccgccgccgccgtcgagccatcaccatccccgctga

SEQ ID NO: 7 - protein, 266 aa, Zea mays
MGRGKVQLKRIENKINRQVTFSKRRNGLLKKAHEISVLCDAEVAVIVFSPKGKLYEYATDSRMDKILERYERYSYAEKALISAESESEGNWCHEYRKLKAKIETIQKCHKHLMGEDLESLNPKELQQLEQQLESSLKHIRSRKSHLMAESISELQKKERSLQEENKALQKELAERQKAVASRQQQVQWDQQTHAQAQTSSSSSSFMMRQDQQGLPPPHNICFPPLTMGDRGEELAAAAQQQPLPGQAQPQLRIAGLPPWMLSHLNA

SEQ ID NO: 8 - DNA, 801 bp, Zea mays
atggggcgcggcaaggtacagctgaagcggatagagaacaagataaaccggcaggtgaccttctccaagcgccggaacggcctgctcaagaaggcgcacgagatctccgtcctctgcgatgccgaggtcgccgtcatcgtcttctcccccaagggcaagctctacgagtacgccaccgactcccgcatggacaaaattcttgaacgctatgagcgatattcctatgctgaaaaggctcttatttcagctgaatctgaaagtgagggaaattggtgccacgaatacaggaaactgaaggccaaaattgagaccatacaaaaatgccacaagcacctgatgggagaggatctagagtctttgaatcccaaagagctccagcaactagagcagcagctggagagctcactgaagcacatcagatcaaggaagagccaccttatggccgagtctatttctgagctacagaagaaggagaggtcactgcaggaggagaacaaggctctgcagaaggaacttgcggagaggcagaaggccgtcgcgagccggcagcagcaggtgcagtgggaccagcagacacatgcccaggcccagacaagctcatcatcgtcctccttcatgatgaggcaggatcagcagggattgccgcctccacacaacatctgcttcccgccgttgacaatgggagatagaggtgaagagctggctgcggcggcgcagcagcagccactgccggggcaggcgcaaccgcagctccgcatcgcaggtctgccaccatggatgctgagccacctcaatgcataa

SEQ ID NO: 9 - DNA, 2554 bp, Zea mays
atcccttcattttagaggaattgaaattcactcaataaagtaacttatttagtttggaatttgacattccaccactttccaaagttcatatattagtctatctcaaattcatggggtgggggatgggaaatgattttatacagtagtagaatttgtttctactctgtaacttacgtgacactcttcatctcactcctctatagtaaaaatatagcacataaatatcttcgatatcttgctaataatagtatacaaatatattttgtataaaactgaattagcttaattgatatatgcctaaattactattattagaatagaattcaattccaatgatccaaacggggcgtaatataaataatacgtaacatccaactgacgtgttcacctatagagaatattccttctgattctactttcagaatgatgccgttgccgtgtatcgagcaagtactctcactcgaagtatcttatctcccacatccagcacaaaaatcttctgttcgtggcaaatcttgtggcggttgaacgaaagaatgctatataagtagctatagagaacgtattatgtgtaaaccaaccgttcagtgtaaatcgtgtgtaaatagtcatgttaattttttggcggcagatcaagtacaaactgtatgcctcggacaaacatgtacaagccacaacactggccactagatctatatccaacgttcataaccatccatccctctttgctgcactctgcaaacgagcacccccatctcgtagcaacatcttgtctccgacaagctctcgatgtagtggaggccctccaccgcaatatcctagtgtatatatgatgttggagaagcgactcctaaataatggtggcaagatgttgctaggtttgtagccatagcctcaatctaagatcatcccaagccatgcgacctgattctacgaggcctacaaccaggcatgacacgtcgtctacccactcttgtgcatcatcggtcacttgatctgacttggttcctaaccacttaccctaggttccaaagccctaagtttctcgtatattgttagtcattcttagtgggagttttatgtgtatttcattcttgttaaatagcatgccaactaagcaaacatgatgacataatatgcaatctaataaaaagatatatgagtgggtttcataaaaaagggagagagtttcatgaggagtgaaactctgaatacagatactgatatgacagctttaaaagtagtgttatgaaatcatcattgagaaatggtattagcactcaatcgatttctacgctgtcaattgtcatgagcacaattttcacccaaagaggcacaccagcaatgtccgcttgtagtgtccgagacgttgctccatcgccgtcgtcttgtttctgtgcgctccattcaatgcggcaagtggctcaatcccaagcggtcgtcgcctcccagccccagcagcaaaatatcttcccatgcggccatgccttgaaaattggaatagattctctagattcaccgccgcgtcatcttcactactttctcactggcccaatcagcatctccttctccgagctcaatcatgctcagtcaagcgtcaccaatggcgtcacggttggttttgtcactgtctgcatgcaagggtatactacgaattttgcttcgcaagtgtaaatggaaaatggatctaaacaactgcactgcaccaattttggaacgcggagccgagagtctgtttgggttcgtttgaaacgcgctgatgtttctcatttttttaatagatgtagttacctgatatttaagttggacgatcaaacgactgtgtcaagtgtgattaacaaaagcatcgaaaataaaatttaacgccataaaaaacagtggataatagtaggacttcataatagaaaaaattatcaaacggaatggaggggcccaacgcagtatatagcagccgggtggtgccggacatccgacgctcgtgccagcaggccattcttctcgccttgtcgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctgaacccgtgcag

SEQ ID NO: 10 - protein, 284 aa, Zea mays
MAAAASTMSLLPISQPRKQQQQGAGAVVVFQRRPWDARRRRYVVPTARLFGPAIFEASKLKVLFLGVDEGEGSSKHLHAHHPAPAPALLPRTYTLTHSDVTASLTLAVSHTINRAQLQGWYNRLQRDEVVAEWKKVRGRMSLHVHCHISGGHLLLDLIAGLRYYIFRKELPVVLEAFVHGDGDLFSRHPELEEATVWVYFHSNLARFNRVECWGPLRDAAAPAPAEDDSTAPAAASIAMEGQMPVGEWPHRCPQQCDCCFPPHSLIPWPNEQDMATAAGQVRQQ

SEQ ID NO: 11 - DNA, 855 bp, Zea mays
atggccgccgccgcttctaccatgtccctgctcccgatctcccagcccaggaagcagcagcagcaaggcgcgggcgccgtggtcgtgttccagcggcggccctgggacgcgcggcggaggcgatacgtcgtcccgacggcgaggttgttcgggccggcgatcttcgaggcgtccaagctgaaggtgctgttcctgggcgtggacgagggcgaggggagctcaaagcatctgcatgctcaccacccggcgccggcgccggcgctgctgccgcggacgtacacgctgacgcacagcgacgtgacggccagcctgacgctcgccgtctcccacaccatcaaccgcgcgcagctgcagggctggtacaaccgcctgcagcgcgacgaggtggtggccgagtggaagaaggtgcgcggccggatgtcgctgcacgtgcactgccacatctccggcggacacttgctcctggacctcatcgccggcctccgctactacatcttccgcaaggagctccccgtggtgctcgaggcgttcgtgcacggcgacggcgacctgttcagccgtcacccggagctggaggaggccacggtgtgggtctatttccactccaacctggcccgcttcaaccgcgtcgagtgctggggtccgctccgcgacgccgccgcccccgcgcccgccgaggacgactccaccgcgccggccgccgcctccatcgccatggagggccagatgcccgtgggcgagtggccgcaccggtgtccccagcagtgcgactgctgcttcccaccgcacagcctcataccctggccgaacgagcaagacatggccaccgccgccggccaggtccgacagcagtag

SEQ ID NO: 12 - protein, 1265 aa, Zea mays
MGISFKLSKVGVRVHPAARSASAAVAEKPGAGGKEGSLSESTREDKRKDVNGIKILPACSKEILPDHEVSFTLSLYERGYLISKSAPMDPSQTSIQDGKTLHPYDRASEKLFSAIEAGRLPGDIFDEIPSKYYNGSVVCEIHDYRKHVSNQAPASSAELGSPIVNKVRLRMTFENVVKDITLLSDDSWSYRDFMEAEACILRALQPELCLDPTPKLDRLYQDPVPHKLSLGIGKKRRLRQNPEVVTSSHMSHGKKVCIDRLPESAKADEMGITSSNAAQQVGGNITIQNMSVSGGSQTLRPNNSSQDAARTLLPQSGLQQTLCYSAAGNDHMAGPPANFSGTSSCISSHQSLIGYSDSVAANSLLSVKREMQDASLQDPKRIKRTGGIDDVQQQQIRPQPLGGQEMQWKNHQLHPQLDVKGMQYASSLSGQRYPSSMMNNMQDPGSSLYFSHQQNLRYDAKQEQMDGSDKSKDALQSMAPETSMLDQQQSQSQHLPQQSVARNNVPNMGQWQNTRFAAEKDFKKEDIIQRRKLAPSSRAPTGPVIQSPVSSKSGELSGSSMGGQFGSAVTSAVTGVQKDKFAANSGTAVGFPSVASSPSDSMHRIQQPAVASSKRKTNSVPKTQPPVSAVGSPASVSNMHALLNASSPSIGTTPMGDQAILDKFVKIDNISHRYQLFNKKKFDKISQKKTIINRNQNVAGCLNSCFHSEDYIDTTRPLCNSMISGTINTCKGRVINFVSTKDMYQGHSRPFPVDFNELSDETVRMQYGDIKDFDDPNSYGCVFILPTKHYADLFAGQLISLMLQDGHSKADDEVVRSTPFANISTPFGPLPNNVVSDVKQEGGVSQQLNAAAHANVAPGTQMQQLPVNRMLPSANGNQILAMQQGYMQGAAMPPRSQHLDQNLVQQPQHQQPQQQPLQQNAQAQVQQPSSLPLNQMQRPQVLPTSPLSQMLGPGSNLPMGSSQIGKNKAPPTSLQLQMLQAQPQQPMSRKVMMGLGSAMNMGNMVNNVVGLGGLGNVMGMGNVRPISSPMASMSGLGNNSNPMNMGMASNLAAAGLRPGMNPAAIAKVRMGLAQQRAAGMYPGMVGMPGSSSSILPSSAGLSMMGQPLNRGNLGPLQRAMMSSMGPPKMPGGNFQLNAQQQIHLQHQLQQLQQNPQQQLQQLQQQQQIQQLQQQQQQQLQQQQLQQQQQMGSPLQQAQVGSPAGSQQSPMMQQQQISPQQMGQQAAMSPQLSSGTLQQMSNNVANPVATPGPPPSPQLSSGQQHS

SEQ ID NO: 13 - DNA, 3798 bp, Zea mays
atggggatctcgttcaagctgtcgaaggtcggggtccgcgtgcacccggccgcgcgctcggcgtccgcggcggtggcggaaaagccgggcgcgggtgggaaggagggttcgctgtctgagtcgacacgcgaggacaaaagaaaagatgtcaatggcatcaaaattttaccagcatgctccaaagaaattttgccagatcatgaggtttctttcacattgagcctctatgagagaggttatctcatttcaaagtcagcacccatggatcctagtcagacctcaattcaggacggcaaaacactgcatccctatgatagagcatcagaaaagttgttctctgctatcgaagctgggaggctacctggcgatatttttgatgagataccaagcaagtactataatggatcagttgtttgtgagatacatgactaccgaaagcatgtgtccaaccaagcgcctgcatcatctgctgagctaggatcaccaattgtgaataaagtacgactgcgaatgacctttgaaaatgttgtaaaggacattacccttctatctgatgattcctggagttacagagattttatggaagctgaggcttgtattttgagagctctacaaccggaactttgcttagaccccacacctaaactggatcgactttatcaggatcctgttccgcataagttgagccttggtatagggaaaaagaggaggctgaggcaaaatcctgaagttgtcacatccagtcacatgtctcatggtaaaaaggtttgcattgataggttacctgaaagtgccaaagctgatgagatgggcatcactagcagtaatgcagctcagcaggttggtggtaacattaccatccaaaatatgtcagtctcaggtggttctcagacacttagaccaaataattcttcacaagatgctgccagaacgcttttgcctcaatctggtctacagcaaaccttatgttattctgctgctggtaatgatcatatggcaggaccacctgccaatttttctggaaccagttcatgcatttcatctcatcagagcctgattggttacagtgactctgtggctgccaacagccttctatctgtgaagagggaaatgcaggatgcctcgcttcaagatcctaagagaataaagcgaactggtggtattgatgatgtacagcagcagcagataaggcctcaaccccttggtgggcaggagatgcaatggaagaaccatcaactgcatccacaattagatgtcaaggggatgcagtatgcatcttcactgagtggtcagagatatccttcttcgatgatgaacaacatgcaagatccaggatcttccttatattttagtcatcagcaaaatttgagatacgatgctaagcaggagcagatggatggttctgataagtcaaaagacgccttgcagtctatggcacctgaaacttccatgctggatcagcagcaatcccaatctcaacatttaccacaacaatcagtggcaagaaataatgttccaaacatgggacagtggcaaaatactcggttcgcagctgagaaggacttcaaaaaagaagacataattcagagaagaaagttagcacctagctctcgtgcccctactgggcctgtgattcagtctccagtgtcctcgaaatctggagagttatcaggcagttcaatgggtggccagtttggttctgctgtgacctcagctgtaacaggggtacagaaagataaatttgctgcaaattccggtactgcagttggatttccttctgtagcttccagtcctagtgactccatgcaccgaatacaacagcctgctgttgcttcctcaaagaggaaaacaaattctgtccccaaaactcaaccgcctgtgagtgctgttgggtctccagccagtgtttcaaacatgcatgcgctgctgaatgcaagcagtccatcgattgggaccacacctatgggagaccaagcaatccttgataaatttgtgaaaattgataacatttcccatcggtaccagcttttcaataagaagaagtttgataaaatatctcaaaagaaaaccattatcaatcgaaaccaaaatgtagctggttgtctcaacagttgtttccattctgaggattatatagataccacaagacctctttgtaattctatgattagtggaactataaacacatgcaagggtagggtaataaactttgtgagcacaaaagacatgtaccaaggtcattcaaggccattcccggttgattttaacgaactgtctgatgaaactgtaagaatgcaatatggagatataaaagattttgatgatccgaattcatatggttgtgtattcatattaccgacaaagcactatgctgacttgtttgcggggcagcttatttcccttatgttgcaagatggacattctaaagctgatgatgaagttgtgcgtagcaccccttttgctaacatcagtacaccctttggacctttaccaaacaacgtagtgagtgatgtaaagcaagagggaggtgtaagccaacaacttaatgccgcagcccatgcaaatgtggcacctggaacacaaatgcaacagcttcctgtcaataggatgcttccatctgcaaatggcaaccagattctagcaatgcagcaaggttatatgcaaggggcagccatgcctccaaggagccagcatcttgaccaaaatttggttcagcagccgcagcaccaacagccacaacagcaaccactgcagcaaaatgctcaagcccaggtgcagcaaccatcatctcttccactgaaccagatgcaaagacctcaggttctgcctacgagcccattatctcagatgttggggcctggctcaaatctcccaatgggctcaagtcagataggtaagaataaggctcctcccacatctttgcagcttcaaatgctacaggcacaaccccaacaacctatgtctaggaaagtgatgatggggcttggctcagccatgaacatgggcaatatggttaacaatgttgttggtcttggtggcctcggaaatgttatgggaatgggcaacgtgcggccaatatcttcccccatggcatcgatgtcaggcttaggtaacaattccaatccaatgaacatgggaatggcatccaatcttgctgcagctggacttcggccaggcatgaaccctgctgctattgccaaggtgcgtatggggttggcacagcaaagggcagcaggcatgtaccctggaatggttggaatgcctggaagcagctcatcaatccttcctagttcagctggcttgtctatgatgggccagccgctaaacagaggcaaccttggccccctccagagggccatgatgtcgtctatgggccctccaaaaatgccaggaggtaactttcagctgaatgctcaacagcaaatacacctccagcatcagttgcagcagctccaacagaacccacagcagcagctccaacagctacagcaacagcaacaaatacaacaactgcagcagcagcagcagcagcagctccaacaacagcaactgcagcagcaacaacaaatgggatctccgttacagcaggcacaggtgggctcacctgctggctcacagcaatcgccgatgatgcagcagcagcagataagccctcagcagatgggacagcaggctgcaatgagcccccagttgagctcaggaactctgcagcaaatgagcaataacgtggccaaccctgtagccactccaggccctcctccaagcccgcagctgagctccggtcaacagcatagctaa

SEQ ID NO: 14 - DNA, 671 bp, Zea mays
cggatccactagtaacggccgccagtgtgctggaattcgcccttgacggcccgggctggtatttcaaaactatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcac

SEQ ID NO: 15 - protein, 514 aa, Zea mays
MTTSTTAKQLRRVRTLGRGASGAVVWLASDEASGELVAVKSARAAGAAAQLQREGRVLRGLSSPHIVPCLGSRAAAGGEYQLLLEFAPGGSLADEAARSGGGRLAERAIGAYAGDVARGLAYLHGRSLVHGDVKARNVVIGGDGRARLTDFGCARPAGGSTRPVGGTPAFMAPEVARGQEQGPAADVWALGCMVVELATGRAPWSDVEGDDLLAALHRIGYTDDVPEVPAWLSPEAKDFLAGCFERRAAARPTAAQPAAHPFVVASASAAAAIRGPAKQEVVPSPKSTLHDAFWDSDAEDEADEMSTGAAAERIGALACAASALPDWDTEEGWIDLQDDHSAGTADAPPAPVADYFISWAEPSDAELEPFVAVAAAAGLPHVAGVALAGATAVNLQGSYYYYPPMHLGVRGNEIPRPLLDHHGDGLEKGQGSHRVCNRETEKVTMKRISLKRRAAFLLDQHHVRSLDKLEYRPRHDRMLRRRQSIYRSNSVLGYDVSKGRQVRWRRAVCIAVAA

SEQ ID NO: 16 - DNA, 1545 bp, Zea mays
atgacgacgtcgaccacggcgaagcagctccggcgcgtgcgcacgctcggccgcggcgcgtcgggcgccgtggtgtggctggcctccgacgaggcctcgggcgagctggtggcggtcaagtcggcgcgcgccgccggggccgcggcgcagctgcagcgcgagggccgcgtcctccggggcctctcgtcgccgcacatcgtgccctgcctcggctcccgcgccgcggcgggcggcgagtaccagctcctgctggagttcgcgccgggcgggtcgctggccgacgaggccgccaggagcggcgggggccgcctcgcggagcgcgccatcggcgcctacgccggggacgtggcgcgcgggctggcgtacctccacggccggtcgctcgtgcacggggacgtcaaggcccggaacgtggtcatcggcggcgacgggcgcgccaggctgaccgacttcgggtgcgcgaggccggccggcgggtcgacgcgccccgtcgggggcaccccggcgttcatggcgcccgaggtggcgcgcggccaggagcagggccccgccgccgacgtctgggcgctcgggtgcatggtcgtcgagctggccacgggccgcgcgccctggagcgacgtggagggcgacgacctcctcgccgcgctccaccggatcgggtacacggacgacgtgccggaggtgcccgcgtggctgtcgcccgaggccaaggacttcctggccggctgcttcgagcgccgcgccgccgcccggcccacggccgcgcagcccgcggcgcacccgttcgtcgtcgcctccgcctccgccgccgccgccatccgcggcccggcgaagcaggaggtggtcccgtcacccaagagcacgctgcacgacgcgttctgggactcggacgccgaggacgaagcggacgagatgtcgacgggcgcggcggccgagaggatcggggcattggcgtgcgccgcctccgcgctgcctgactgggacaccgaggaaggctggatcgacctccaggacgaccactcggccggaactgccgacgcaccgccggcgcccgtcgcggactacttcatcagctgggcggagccgtcagacgcagagctggaaccattcgtcgccgtcgccgccgccgcaggtctcccgcacgttgcaggagttgcattagcaggcgccaccgccgttaacctgcagggcagttattattattacccgcctatgcatctaggcgtccgcggaaacgagattccacgcccgttgttggatcatcatggcgacgggttagaaaaggggcagggatcccaccgcgtttgtaacagagaaacagaaaaggtaacaatgaaacgaatttcgttaaaaagaagagctgctttccttctcgaccagcatcacgtgcgatcgctggacaaactggaatatcgtccacgtcacgaccgaatgctgcgtcgacggcaatctatatatcggagcaatagcgtccttggttacgacgttagcaaaggtaggcaggtccgttggcgccgtgcggtttgcattgccgttgctgcctga

SEQ ID NO: 17 - DNA, 1442 bp, Zea mays
agcttggtaccgagctcggatccactagtaacggccgccagtgtgctggaattcgcccttctgggcaagctgtcactaggactggacaaaatactcgtggctcgataactcgctcgactcgtctcgttagtagctcagctcgactcggctcgttttaattttgtagcgagccaagctagcattctagctcgattctctaatgagccagctcgggttagctcgtgagctagctcgcgagccaaacgagctaagccacaacacaaatttgtctagtcattgatgtcgtctcatctctcatagtcttgttttctcgtagttatgatctgtgatatggacatgtgtggatgtgccatgtacttaaatatttatattattgcatggctacatgtttgtagtgttaaatacttaaaatataatttttcggttataaatatatttatgtacatagatatttatatttagttgtgtggctcacgagcctaacgagctggctcgagcttcctaacgagccgagccgagccagctgtttagcccgttagtataacgagccgagccgagctggctcgttatagtaacgagtcataacgagccgagccataacgagccaagctggttcgatatccacccctagctgtcaccgtcgcccagtccgcttcgttcggtcagcgggccccgcctcatctgcattcttccattctcgtcctccgacctcatctgcattttcccagccaagtagtaggtaaactagtggcggtcccgtggccgtggcatcaggaaaagaatatgccgtcccagcccaccatccccccaccgtcccgaaattccagaactaccctcggctccagctataaatagccgcccccgggagacgttcgaaaccttccccatctccggataaaagataaggagtgtctctcctctctttcagctaagtccctgccccctctctttttcttacattcaggtcctcgcagctcctctcttttttcttgtttctttctttcgatctgcgagccgtccaggtccagtactctcctttccgtgaaggaactcttgcagccggcccctctggtttcctcgaattcttgttccccggtccctcctcctgtccccgcgtagatccgtccgtccgaggagcacaccgtccccacccccatgtttacccaccagttcctctgacggccgccgtgctccgatgaagctgagcgtgctccgtatccgccgctcccactccttctccgtcgccttcctctactggttctacgtcttctcatgaacgcatcgcccctctccacctgctgatccttcgccgtctctctctctctctctctctctctctcttagatagtcttttgaatccatctctagggctcttgtttctccccatcctccccccacccacccccccaccaaacagattcaatccgacaagacaagcatcc

SEQ ID NO: 18 - protein, 224 aa, Triticum aestivum
MEQCGVGLYGVVEGSGYATVTTAPPKRPAGRTKFRETRHPLYRGVRRRGAAGRWVCEVRQPNKKSRIWLGTFATPEAAARAHDVAALALRGRAACLNFADSATLLAVDPATLRTPQDIRAAAIALAQAACPHDARRSSVSVASARAPAMVIMEEAAAAPYDSYAMYGGLADLDQHSYCYSNGMSGGGDWQSISHMDGADEDGSYGAGDVALWSYWSRGIDRADC

SEQ ID NO: 19 - DNA, 675 bp, Triticum aestivum
atggaacagtgcggcgtgggcctctacggcgtcgtcgagggcagcggatacgcgacggtgactaccgcgccgcctaagcggccggcggggcggaccaagttccgggagacgcgccacccgctctaccgcggcgtgcgccggcgcggcgccgcggggcggtgggtgtgcgaggtgcgccagcccaacaagaagtcgcgcatctggctcggcaccttcgccacgcccgaggccgccgcgcgcgcccacgacgtcgccgcgctcgcgctccggggccgagccgcctgcctcaacttcgccgactcggccacgcttctcgccgtcgaccccgccacgctccgcacgccccaggacatccgcgccgccgcaatcgcgctcgcccaggcggcctgcccgcacgacgcgaggaggtcctctgtgtccgtggcgtccgcgcgggcgcccgcgatggtgatcatggaggaggccgcggcggcaccgtacgacagctacgccatgtacggcggcttggcggacctggaccagcattcctactgctactccaacgggatgagcggcggcggcgactggcagagtatctcgcatatggacggagccgacgaagacggcagctacggcgcaggagacgtcgcgctctggagctactggtcgcgtgggatcgatcgggcagattgttga

SEQ ID NO: 20 - protein, 216 aa, Arabidopsis thaliana
MDSFSAFSEMFGSDYESPVSSGGDYSPKLATSCPKKPAGRKKFRETRHPIYRGVRQRNSGKWVCELREPNKKTRIWLGTFQTAEMAARAHDVAAIALRGRSACLNFADSAWRLRIPESTCAKEIQKAAAEAALNFQDEMCHMTTDAHGLDMEETLVEAIYTPEQSQDAFYMDEEAMLGMSSLLDNMAEGMLLPSPSVQWNYNFDVEGDDDVSLWSY

SEQ ID NO: 21 - protein, 305 aa, Zea mays
MKFGKSLSGQIVETLPEWRDKFLSYKDLKKRLKLIGAGNGAERQPKRARRDDSGEADAAAAAAAMTPEEAEFMQLLEAELDKFNSFFVEKEEEYIIRQKELQDRVARAAGRESKEELMRVRKEIVDFHGEMVLLENYSALNYTGLVKILKKYDKRTGALIRLPFIQKVLQQPFFTTDLLYKLVKQCEAMLEQLLPVSEASVSSEDGKGDSNDEEKLAKPSSSLVNGGGMPELDEIEYMESMYMKSTVAALRSLKEIRGKSSTVSMFSLPPLQGNNAQDSYQIRAEQLDEEPERWSKVTVIEQAAK

SEQ ID NO: 22 - DNA, 918 bp, Zea mays
atgaagttcggcaagagcctgagcggacagatcgtggagacgctcccggagtggcgcgacaagttcctgtcctacaaggatctcaagaagcgcctcaagctcatcggcgccgggaacggggctgagcggcagccgaagcgggcccgccgcgacgactccggggaggctgacgcggccgcggccgcggcggcaatgacgccagaggaggcggagttcatgcagctcctggaggctgagctcgacaaattcaactcttttttcgtcgagaaggaggaggagtacatcatccgtcagaaggagctgcaggaccgggtggccagggcggccgggcgggaatccaaggaggagctcatgcgggtgcgcaaggagatcgtcgacttccacggcgagatggtgctgctcgagaactacagcgccctcaactacacaggattggttaagattctcaagaaatatgacaagagaactggagccctgatccggctgcctttcattcagaaagttcttcagcagcctttcttcaccactgatctcctgtataagcttgtgaagcagtgtgaagccatgctggagcagctccttccggttagtgaggcatctgtctcaagtgaagatggaaaaggagactctaatgatgaagagaagctggcgaagccaagttcttccttggtaaatggtggtggcatgccagagttagatgagatcgagtacatggagagtatgtacatgaagagcaccgtcgcggcccttaggtcactgaaggagatccggggcaagagctctactgtcagtatgttctcgttgccacctcttcagggcaacaatgcacaggacagctaccagatccgggcagagcaactagatgaggagccggagaggtggagcaaagtaacggtgatagagcaggcggccaaatga

SEQ ID NO: 23 - protein, 494 aa, Zea mays
MAPPPSMPAASDRAGPGRDAGDSSSLRLRRAPSADAGDLAGDSSGGLRENGEPQSPTNPPPQEQQQHEMLYYRASAPAHRRVKESPLSSDAIFRQSHAGLLNLCIVVLIAVNSRLIIENLMKYGLLIRAGFWFSARSLGDWPLLMCCLTLPVFPLVALMAEKLITRKLIGEHVVILLHIIITTSAIVYPVVVTLKCDSAVLSGFVLMFLASIMWMKLVSYAHTNYDIRVLSKSTEKGAAYGNYVDPENMKDPTFKSLVYFMLAPTLCYQPTYPQTTCIRKGWVTQQLIKCVVFTGLMGFIIEQYINPIVKNSKHPLKGNFLNAIERVLKLSVPTLYVWLCMFYCFFHLWLNIVAELLCFGDREFYKDWWNAKTVEEYWRMWNMPVHKWIIRHIYFPCIRKGFSRGVAILISFLVSAVFHEICIAVPCHIFKFWAFSGIMFQIPLVFLTRYLHATFKHVMVGNMIFWFFFSIVGQPMCVLLYYHDVMNRQAQASR

SEQ ID NO: 24 - DNA, 1485 bp, Zea mays
atggccccgcccccctccatgcctgccgcctccgatcgcgccggccctggccgcgacgcgggcgactcgtcctcccttcgcctccgccgcgccccctcagccgacgccggcgaccttgccggcgattcctcgggaggcttgcgggagaacggcgagccgcaatcgccgacgaatccgccgccgcaggagcagcagcagcacgagatgctatactaccgcgcgtcggcgcccgcccaccgccgcgtcaaggagagccccctcagctctgacgccatcttccggcagagccatgctggtcttctgaatctatgcattgttgttctgatcgcagtgaacagcagactcattattgagaatttaatgaagtatggcctgttgataagagctggattttggtttagtgcaagatcgctgggtgactggccccttctaatgtgctgcctcactctaccagttttcccactagttgcactcatggctgagaagctgatcacaagaaagctcattggtgaacatgtggttattctactccatatcattattacaacatctgccattgtctatccagttgttgtgactcttaagtgtgactcagcagtactatctggatttgtgctaatgtttcttgcgagcatcatgtggatgaagcttgtctcttatgcacatacaaattatgatataagggtattgtccaaaagtactgaaaagggtgctgcatatggaaattatgtcgatcctgagaatatgaaagatccaacctttaaaagtctagtgtactttatgttggccccaacactttgttaccagccaacttatcctcaaactacatgtattagaaagggttgggtgacccagcaactcataaagtgcgtggtttttacaggcttgatgggcttcataattgagcaatatataaacccaattgtgaagaattccaaacatccactgaaagggaattttttgaatgctatagaaagagtcttaaaactctcagtgccaacattatatgtatggctttgcatgttctattgcttttttcatttatggctgaacattgtagctgaactcctctgtttcggtgaccgtgaattctataaggactggtggaatgccaaaactgttgaagagtactggaggatgtggaacatgcctgttcataagtggatcatcagacacatatattttccatgtataaggaaaggcttttccaggggtgtagctattctaatctcgtttctggtttcagctgtatttcatgagatatgtattgcggtgccttgccacattttcaaattctgggcattttctgggatcatgtttcagatacccttggtattcttgacaagatatctccatgctacgttcaagcatgtaatggtgggcaacatgatattttggttcttcttcagtatagtcggacagccgatgtgtgtccttctatactaccatgacgtcatgaacaggcaggcccaggcaagtagatag

SEQ ID NO: 25 - DNA, 879 bp, Zea mays
aattcccatgatcttctctccttcatcaatggatgccatgtttcataacaataacaccaaatgtttgatgagctaccaacaattgcgcaaagactatggctaagctcgagctcgctcgctacaagttgttgactttcaaatacaagtttgtttttggaacaccaaatattctacatgatctttcactaagttgcgcaccactatcaaaagattatctaggccattattcaagtaaagagtgaacacgtctaagacccacaaccacaccaaatagaatacgcatacatgcaacatattgtgcaagaagtatccaactggactcccatgtattctaaaactattttcgtagagttaaagttatgacaaacttatcaaataaaaatttgaacgctggaccaaaactttcatctttcaaatccaccatcgtctatcctcataaattgttttgattataacacatctacgtaaatcatttgttttgaacaatactaatttaattttattaagtcaaataacctgcttagaaaataatccctccacctcatttaacaatttcttgtcaaacacacaccaagaaaaaaattaatgaaagagaaaagaaatgaaaaggacatggagttgaatactagcaaaattgattgaaggaagattcacaattgaaattgaaaccatttaatttattttcgggtccataataataaattggtaagaataaaaacccgatcaagtccggtacagtacaattccactccaccaactccttacttaaacccctatttatacccactctcatcctcactcttccttcacctctcacactctcttctctctctcaaaaccctcacacaaacgctgcgtttagtgtaagaaattcaatcc

SEQ ID NO: 26 - protein, 525 aa, Zea mays
MLSELNAPPAPLPPATPAPRLASTSSTVTSGAAAGAGYFDLPPAVDSSSSTYALKPIPSPVAAPSADPSTDSAREPKRMRTGGGSTSSSSSSSSSMDGGRTRSSVVEAAPPATQASAAANGPAVPVVVVDTQEAGIRLVHALLACAEAVQQENFSAAEALVKQIPMLASSQGGAMRKVAAYFGEALARRVYRFRPPPDSSLLDAAFADLLHAHFYESCPYLKFAHFTANQAILEAFAGCRRVHVVDFGIKQGMQWPALLQALALRPGGPPSFRLTGVGPPQPDETDALQQVGWKLAQFAHTIRVDFQYRGLVAATLADLEPFMLQPEGDDTDDEPEVIAVNSVFELHRLLAQPGALEKVLGTVRAVRPRIVTVVEQEANHNSGTFLDRFTESLHYYSTMFDSLEGAGAGSGQSTDASPAAAGGTDQVMSEVYLGRQICNVVACEGAERTERHETLGQWRSRLGGSGFAPVHLGSNAYKQASTLLALFAGGDGYRVEEKDGCLTLGWHTRPLIATSAWRVAAAAAP

SEQ ID NO: 27 - DNA, 1578 bp, Zea mays
atgctgtccgagctcaacgcgcccccagcgccgctcccgcccgcgacgccggccccaaggctcgcgtccacatcgtccaccgtcacaagtggcgccgccgccggtgctggctacttcgatctcccgcccgccgtggactcgtccagcagtacctacgctctgaagccgatcccctcgccggtggcggcgccgtcggccgacccgtccacggactcggcgcgggagcccaagcgaatgaggactggcggcggcagcacgtcctcctcctcttcctcgtcgtcatccatggatggcggtcgcactaggagctccgtggtcgaagctgcgccgccggcgacgcaagcatccgcagcggccaacgggcccgcggtgccggtggtggtggtggacacgcaggaggccgggatccggctcgtgcacgcgctgctggcgtgcgcggaggccgtgcagcaggagaacttctctgcggcggaggcgctggtcaagcagatccccatgctggcctcgtcgcagggcggtgccatgcgcaaggtcgccgcctacttcggcgaggcgcttgcccgccgcgtgtatcgcttccgcccaccaccggacagctccctcctcgacgccgccttcgccgacctcttacacgcgcacttctacgagtcctgcccctacctgaagttcgcccacttcaccgcgaaccaggccatcctcgaggccttcgccggctgccgccgcgtccacgtcgtcgacttcggcatcaagcaggggatgcagtggccggctcttctccaggccctcgccctccgccctggcggccccccgtcgttccggctcaccggcgtcgggccgccgcagcccgacgagaccgacgccttgcagcaggtgggctggaaacttgcccagttcgcgcacactatccgcgtggacttccagtaccgtggcctcgtcgcggccacgctcgccgacctggagccgttcatgctgcaaccggagggcgatgacacggatgacgagcccgaggtgatcgccgtgaactccgtgttcgagctgcaccggcttcttgcgcagcccggtgcactcgagaaggtcctgggcacggtgcgcgcggtgcggccgaggatcgtgaccgtggtcgagcaggaggccaaccacaactccggcacgttcctcgaccgcttcaccgagtcgctgcactactactccaccatgttcgattctctcgagggcgccggcgccggctccggccagtccaccgacgcctccccggccgcggccggcggcacggaccaggtcatgtcggaggtgtacctcggccggcagatctgcaacgtggtggcgtgcgagggcgcggagcgcacggaacgccacgagacgctggggcagtggcgcagccgcctcggcggctccgggttcgcgcccgtgcacctgggctccaatgcctacaagcaggcgagcacgctgctggcgctcttcgccggcggcgacgggtacagggtggaggagaaggacgggtgcctgaccctggggtggcatacgcgcccgctcatcgccacctcggcgtggcgcgtcgccgccgccgccgctccgtga

SEQ ID NO: 28 - protein, 526 aa, Sorghum bicolor
MGYNGLLPLMLLAAGWCAVAAALVLAISAWLQRPRRVAEAFRRQGIDGPPPSSFLSGNLSEMQARAAAAAVAEAAGGRDFQKEGFDDYCKKIFPYFEKWRKAYGETYLYWLRRRPALYVSDPELIREIGRCVSLDMGKPTYLQKGQEPLFGRGVLKANGAEWHRQRKLIAPEFYMAKVKGMVELMVDAAQPLLASWEDKVAAAPGGVAEIDVDEDIRSFSFDVISRACFGGDYSRGREIFLRLRALSGLMSETSVIFTIPSLRHLPTKKNRRIWKLTHEIRSLILQLASERKAAAAPTPGRDFLGSIIDSSRDQPRADDFVVDNCKNIYFAGHETSAVTATWCLMLLAAHPEWQDRARAEALDVCGGDAAAPDFDAVARMRTLHAVVLETLRLFPPSSFVVREMFRDMQLGTRLRAPKGTYLFVPVSTMHHDAAVWGATARRFDPGRFRDGVAAACKHPQAFMPFGLGARTCLGQNLALVEVKALVALVLARFSLALSPDYRHAPAFRFIIEPEFGLRLRVHRLGH

SEQ ID NO: 29 - DNA, 1581 bp, Sorghum bicolor
atggggtacaacggcctcttgccgctgatgctgctggcagctgggtggtgcgccgtcgcggcggcgctcgtcttggccatttccgcgtggctgcagcggccgcgccgcgtcgcggaggccttccgtcggcagggcatcgacggcccgccgccgtcgtcgttcctgtcgggtaacctctcggagatgcaggcgagggcggccgccgcggcggtggcggaggccgccggcggccgggacttccagaaggaaggcttcgacgactactgcaagaagatcttcccctacttcgagaagtggaggaaagcctacggcgagacatacctgtactggctacgccgccggccggcgctgtacgtgtcggacccggagctgatccgcgagatcgggcgctgcgtgtcgctggacatgggcaagcccacctacctgcagaaggggcaggaacccctcttcggccgcggcgtcctcaaggccaacggcgccgagtggcatcgccagcgcaagctcatcgccccggagttctacatggccaaggtcaagggcatggtggagctgatggtggacgcggcgcagccgctgctggcgtcgtgggaggacaaggtcgccgcggcgccgggcggcgtcgcggagatcgacgtggacgaggacatcaggagcttctccttcgacgtcatctccagggcctgcttcggcggcgactactccagggggcgggagatcttcctccgtctcagggcgctgtcgggcctcatgtccgagaccagcgtcatcttcaccatcccgtcgctcaggcaccttcccacgaagaagaaccggaggatctggaagctcacgcacgagatccggtcgctgatcctgcagctggcgagcgagcgcaaggcagcggcggcgccgacgcccggccgcgacttcctgggctccatcatcgacagcagccgtgaccagccgcgcgcggacgacttcgtggtggacaactgcaagaacatctactttgcgggccacgagacgagcgcggtcaccgcgacgtggtgcctcatgctcctcgccgcgcacccggagtggcaggaccgcgcgcgcgccgaggcgctcgacgtctgcggcggcgacgccgccgcgccggacttcgacgcggtggccaggatgaggacgctgcacgcggtggtgctggagacgctgcgcctcttcccgccgtcgtcgttcgtggtgcgggagatgttccgcgacatgcagctcggcaccaggctgcgcgcgcccaagggcacctacctcttcgtgccggtctccaccatgcaccacgacgccgccgtctggggcgccaccgcgcgccggttcgacccaggaaggttccgcgacggcgtggcggccgcgtgcaagcacccgcaggcgttcatgcccttcggcctcggcgcgcgcacctgcctcggccagaacctcgcgctcgtcgaggtcaaggcgctcgtggcgctcgtcctcgcccgcttctcgctcgcgctgtcgccggactaccggcacgcccccgcgttccggttcatcatcgagccggagttcggcctgcgcctccgcgtgcaccgcctcggccactga

SEQ ID NO: 30 - protein, 399 aa, Zea mays
MMNLSAAANGRDEFPPYVVPSNAAAPPPSLLPTMEQQQESSIHREHHQLLGYNLEANSLALLPPSNAAAAHHHTTFAGGHSPHDILHFYTPPPSAASHYLAAAAGNPYSHLVSAPGTTFHQTSSSYYPPAAAAQAAPEYYFPTLVSSAEENMASFAATQLGLNLGYRTYFPPRGGYTYGHHPPRCQAEGCKADLSSAKRYHRRHKVCEHHSKAPVVVTAGGLHQRFCQQCSRFHLLDEFDDAKKSCRKRLADHNRRRRKSKPSDADAGDKKRAHANKAAAAKDKAESSSKNMDIGDGLGAQILGSALLSKEQDQTMDLGEVVKEAVDPKGKASMQQHYGFPFHSSSAGSCFPQTQAVSSDTTSNIGQVQEPSLGFHHQHHQHSNILQLGQAMFDLDFDH

SEQ ID NO: 31 - DNA, 1200 bp, Zea mays
atgatgaacctatcggctgccgccaacggccgcgacgagttccccccctacgtcgtgccgtccaacgcggccgctccgcccccttccctgctcccaaccatggagcagcagcaggagagcagcatccacagggagcatcatcagctgctgggctacaacctcgaggccaactcgctggccctcctgcccccgtccaacgccgccgccgcccaccaccacaccaccttcgccggcggccacagcccccacgacatcctccacttctacacacctcctccttccgccgcctcgcactacctcgccgccgccgccggcaacccctacagccacttagtctccgcgcccgggaccaccttccaccagacctcgtcgtcctactacccgcccgcggcggcggcgcaggccgcgcccgagtactacttccccaccctcgtcagctccgccgaggagaacatggccagcttcgccgccacgcagctcggcctcaacctcggctaccgcacctacttcccgcccagaggaggctacacgtacggccaccacccgccgcgctgccaggccgagggctgcaaggccgacctctccagcgccaagcgataccaccgtcgccacaaggtgtgcgagcaccactccaaggcgcccgtcgtcgtcaccgccggtggactgcatcagaggttctgccagcagtgcagcagattccatctgctggatgagttcgacgatgctaagaagagctgcaggaaacgcctagcggaccacaaccgccgccgccggaagtcaaagccatcggatgctgatgccggagacaagaaaagggcacatgcgaacaaagcagctgcagctaaagacaaagcagagagtagcagcaagaacatggatatcggagatgggttaggcgcacagatactgggaagtgcactcttgtccaaggaacaagatcaaaccatggatcttggagaggtggtgaaagaagcagtggatcccaaggggaaggcatcaatgcaacagcattacggcttccccttccattcgtcgtcagcaggatcttgcttcccccagacccaagccgtctccagtgataccacatccaatataggtcaagtgcaagagccaagcttagggttccaccatcagcaccaccaacacagcaacatcttgcagctcggccaggctatgtttgatctcgacttcgatcactag

SEQ ID NO: 32 - DNA, 1319 bp, Zea mays
agcttgagtgttgtcgtgttgctcgattgctaacaccctccctcctcgaacagcgcccgacatctcttaaggtagtgtttggttctggagttaggtggggtggagtcgttccattctactttttgtgttgttgagttgtattccggttgaagcagagtggctcaaattctagaatatacccttgagatgcggcatcccgtggctcctcaaaattaatcggatttggctgctcgtgagccgagcctcccattcccatcccatctcttccctgatgctcgacataagctcacccaagtttgtttcaattttcttctatagataaattacttggggcagtttagaatttgcacgttgtttatgtgagactacgtctattagctatttcttaattataaaactcaatgcctactgatatgtatgatataagtatgtttttattgatcattgagttaacatatgagaaacgaaaaaaaatatactctattttgtatttatcaaccaaatattagatgaagtcattgtaaaattaaattatcaaacatagaacagagtgactttgttatcaaaatctataatggagccatttcatctaaatgagcaccagaaccgaacactgctcagagttccaagacaaggtgtcccggcccaatgagtcgcctgcaactgtaatcgagtggttgggcttgggcccgagggcctatcggccattcatcatcaccgtctctctttgcctgggccgctccaatgtgacatgacctgatgtgacgcgacgtgatacgatcccaccgcgcggcgcggagcacacgggtggctagtagtgtagtagggcccggcagggcatcttttctgtgggcctgtggctggtgcagggagagagatgaggtaccggcgctgagtcgctgacgggtggggcccgggtcggtgccgaaggagggggtggggtggtggcgcgtccatcccacgcgactctcccacacaaataccatcaccttcgctaccattgcttcaccatcaccaccaccccgcggctgcagctcagcagctccagacctcaccagaggcacctaccacaccgcccgccgccgatcccgtcacccgtctcctccccgctgcggagcgtctccagccctgcccggtgcccgcccagatggtaagcacgcggcaccacacttcacctccagtcctgttcatcggctatgcgatctgcattttcgtttgctcgatcgacggatctgccatgctcttcttcttcacctccctcgtcttcgtctccactgcctctgaatcttgtttcgcctcctctcccccgggttctgcag

SEQ ID NO: 33 - protein, 506 aa, Zea mays
MGAMMASITSELLFFLPFILLALLALYTTTVAKCHGTHPWRRQKKKRPNLPPGARGWPLVGETFGYLRAHPATSVGRFMERHVARYGKIYRSSLFGERTVVSADAGLNRYILQNEGRLFECSYPRSIGGILGKWSMLVLVGDAHREMRAISLNFLSSVRLRAVLLPEVERHTLLVLRSWPPSDGTFSAQHEAKKFTFNLMAKNIMSMDPGEEETERLRLEYITFMKGVVSAPLNFPGTAYWKALKSRASILGVIERKMEDRLEKMSREKSSVEEDDLLGWALKQSNLSKEQILDLLLSLLFAGHETSSMALALAIFFLEGCPKAVQELREEHLLIARRQRLRGASKLSWEDYKEMVFTQCVINETLRLGNVVRFLHRKVIRDVHYNGYDIPRGWKILPVLAAVHLDSSLYEDPSRFNPWRWKSNNAPSSFMPYGGGPRLCAGSELAKLEMAIFLHHLVLNFRWELAEPDQAFVYPFVDFPKGLPIRVQRVADDQGHRSVLTESTRG

SEQ ID NO: 34 - DNA, 1521 bp, Zea mays
atgggcgccatgatggcctccataaccagcgagctcctcttcttccttcccttcatcctgctggccctcctcgccttgtacaccaccaccgtcgccaaatgccacggcacccacccgtggcgccgtcagaagaagaagcggcccaacctgcccccgggcgcccgcggatggcccttggtcggcgaaactttcggctacctccgcgcccacccggccacctccgtgggccgcttcatggagcggcatgtcgcacggtacgggaagatataccggtcgagcctgttcggggagcggacggtggtgtcggcggacgcggggctgaaccgctacatcctgcagaacgaggggcggctgttcgagtgcagctacccgcgcagcatcggcggcatcctgggcaagtggtccatgctggtgctcgtgggcgacgcgcaccgcgagatgcgcgctatctcgctcaacttcctcagctccgtccgcctccgcgccgtgctgctccccgaggtggagcgccacaccctgctggtcctccgctcgtggccgccctccgacggcaccttctccgcccagcacgaagccaagaagttcacgtttaacctgatggcgaagaacataatgagcatggaccccggcgaggaggagacggagcggctgcggctggagtacatcaccttcatgaagggcgtcgtgtcagcgccgctcaacttcccgggcacggcctactggaaggcgctcaagtcgcgcgcgtccatacttggagtgatagagaggaagatggaggacaggcttgagaagatgagcagggagaagtcaagcgtggaggaggacgaccttcttggatgggccctgaagcaatccaacctgtccaaggaacagatcctggacctcttgctgagcctgctcttcgcggggcacgagacttcgtccatggcgctcgccctcgccatcttcttcctcgaagggtgccctaaggccgtgcaagaactccgggaggagcatctcctgattgctaggagacaaaggctaaggggggcgtccaaattgagctgggaagactacaaggaaatggttttcacgcagtgtgttataaacgagacattgcggctcggcaacgtggtcaggttcctgcaccggaaggtcatccgagatgtacactacaatgggtacgacataccgcgggggtggaaaatcctgccggttctagcggcggtgcacctggactcgtcgctgtacgaggaccccagccggttcaacccttggagatggaagagcaacaacgcgccaagcagcttcatgccgtacggcggcgggccgcggctgtgcgccgggtcggagctggccaagctggagatggccatcttcctgcaccacctggtgctcaacttccggtgggagctggcggagccggaccaggccttcgtctaccctttcgtcgacttccccaagggcctcccgatcagggtccagcgggtcgccgacgaccaaggccatcgtagcgttttgaccgagagcacaagaggctga

SEQ ID NO: 35 - DNA, 1722 bp, Zea mays
aacgaacctctatcaaacaagcagtcaggtcgcggccaatcacggtcatgggtggattgggcctgggtgaggtaggccgtaggcccgtagcagcgaacggctctgatagcactgttgctgtagacctgtagccgccgcatcgtgctcgtgcagcagcagcaggactgcaggagacacaggtgtcccgcattggcccgtccccgtcgtggcctggccgctgccgtcggaccggcccaaaagtgggcggccccttgctgcgtcagcccgccccacgcgttgtccgcctctccggactgcgaaaagtgaccgagccgggaaaccaggcgaccagttgctgccccttccccgttttgcccaaacattaccccgcagacttcatcacgtgcacggcgtacggtgcgcttaaaaaagcaaaataaaaataaaaaccccgacgagccgcatactccaaacaatctgatatctttaatgcttgggcaccgctgcacgtggcaccagcacggccgtgcaaattaatttgaaagcaaaacattccgatacaaaaaatgacaacaacgaatctagttagatctatttaaaacaatacctccattctcgaatatttatcgtccactagttcagtttttaacgcgcgcgacaaataaaaaagaactaagaaagtatactactccactaccatcctaatgcctactttgagtaatctagtattgatcctaatccatatgtattaaggtggtttgcagtataacttaaactaatttacatataatccacctcaacacatatggattatggtcaatactagagtatccaaacaaagcttaaggattgtttggtttctaactattttttaatccatccattttattttattttagaccctaaactaataaatacgataactaaaatagaacccaaacacccatagagactaaacaccccctaaaacaagtcttgttctcatatgttagagtcaaacgtttttatttttaataaaacatataaaaataacactaatagttttaatatataaatagaattaatagatggagcgttggtttttttgtgtaataaatttattcgaagatactactgctaatactttggtcgaaaataaatgttatagcgatattcattatagctaccagtagtcgagtggagtagatagaaacaaaaacagataatagctgctgcctgccagcatcttgtcgtcattacttcatgccaggcaaggtgtgtgagagaagccggtttcgaccgtacgaggaaagaccctggcagcgggcccttgcgatgagagatgccgtggggccaggtgggcccgggcacgccgcatcggccaatgccagttcgacagcggcggcggggaaaccatcccggtttcgctataccccctccccctccgatccgtcgcggcagcgctcatcaccgctttaaatccgcctcctcccagcgtctccctcctcccggcctgtcccctcacctccctccccctatctctccaccgccgcagctagctgcgacgtcatgcactcgccggcgccaccgccaccgcatactatctacaattagccagccgtaggcttacctatcctgtgtcaagcaagcctctcgcaagcaacaaggaaggaagctagctagttttatagctgctgtcggcggcggcggctgaagcgacgtgcctgagctaggatttaggttgagatcaggagagggagaagg

SEQ ID NO: 36 - DNA, 638 bp, Zea mays
tctgcctcatcaacagctgcagtatttgctagccacatatatatacacagttcgacacgtagttataacggaagagagaagcaaagagagaggcagagtgactgcaaccatcagtagttctatgattttattttttaccgttttgttgctgtttcatggtgtttatttgattgtagggtggaggagaggtgaaagctgacagaagagagtgagcacacatggtgcctttcttgcatgatgtatgatcgagagagttcatgctcgaagctatgcgtgctcacttctctctctgtcagccattagaactcctctatctctcaatctcgatctccctctttctttgttgatctctcccatggtgatatttatttgcttcctacgtgttgtgttctctttcttcagcacacacacaacctgttcatgttaccttagggttaaagtttttgcactttgcgtgaagatggaaagacaaacagtagatgagttttttgaaggtttgacagaagagagtgagcacacacggtggtttcttaccatgagtgtcatgctaggagctgtgcgtgctcaccctctatctgtcagtcactcatcaagcccatctgtcttattagcttgtttccgctgctaataaatattct